(19) United States
(12) Patent Application Publication
(10) Pub. No.: US 2018/0368968 A1
(43) Pub. Date: Dec. 27, 2018

(54) SYSTEM FOR CREATING A GRAFT DEVICE

(71) Applicant: NEOGRAFT TECHNOLOGIES, INC., Taunton, MA (US)

(72) Inventors: Cory LEESON, Lakeville, MA (US); Matthew MANNARINO, Waltham, MA (US); Mohammed EL-KURDI, Mansfield, MA (US); Jon MCGRATH, Duxbury, MA (US)

(21) Appl. No.: 16/063,503

(22) PCT Filed: Dec. 20, 2016
(86) PCT No.: PCT/US2016/067879

§ 371 Date: Jun. 18, 2018

Related U.S. Application Data
(60) Provisional application No. 62/270,215, filed on Dec. 21, 2015.

(51) Int. Cl. A61F 2/06; A61M 1/36; A61L 27/50; A61L 27/16; A61L 27/18; A61L 27/36; D01D 5/00; D01D 13/02

(52) U.S. Cl. CPC A61F 2/062; D10B 2509/06; A61F 2/064; A61L 27/507; A61L 27/16; A61L 27/18; A61L 27/3604; A61L 27/3625; A61L 27/3629; D01D 5/0038; D01D 5/0076; D01D 13/02; A61F 2240/001; A61M 2207/00; A61M 2207/10; A61M 1/3655

(57) ABSTRACT

Provided are systems for applying a polymer fiber matrix to a tubular conduit to create a graft device. The system comprises a polymer solution comprising at least one polymer and at least one solvent; a polymer delivery assembly constructed and arranged to receive the polymer solution and to deliver the polymer fiber matrix to the tubular conduit; a rotating assembly constructed and arranged to rotate at least one of the tubular conduit or the polymer delivery assembly; and a controller constructed and arranged to control the polymer delivery assembly and the rotating assembly. The system is constructed and arranged to reduce the amount of the solvent in the graft device. Methods of applying a polymer fiber matrix with reduced solvent are also provided.

### RELATED APPLICATIONS

This application claims the benefit of U.S. Provisional Application No. 62/270,215, filed Dec. 21, 2015, the contents of which are hereby incorporated herein by reference in their entity for all purposes.

This application is related to U.S. patent application Ser. No. 13/502,759, filed Apr. 19, 2012; U.S. Pat. No. 8,992,594, filed Jun. 14, 2012; U.S. patent application Ser. No. 13/997,933, filed Jun. 25, 2013; U.S. Pat. No. 9,445,874, filed Jan. 18, 2013; U.S. patent application Ser. No. 13/979,243, filed Jul. 11, 2013; U.S. patent application Ser. No. 13/984,249, filed Aug. 7, 2013; U.S. Pat. No. 9,155,610, filed Jun. 12, 2014; U.S. patent application Ser. No. 14/354,025, filed Apr. 24, 2014; U.S. patent application Ser. No. 14/378,263, filed Aug. 12, 2014; U.S. patent application Ser. No. 15/023,265, filed Mar. 18, 2016; U.S. patent application Ser. No. 15/036,304, filed May 12, 2016; U.S. patent application Ser. No. 15/108,970, filed Jun. 29, 2016; the content of each of which is incorporated herein by reference in its entirety for all purposes.

### FIELD OF THE INVENTION

This disclosure relates generally to systems for producing graft devices for a mammalian patient, and more particularly to systems for producing graft devices for providing cardiovascular bypass.

### BACKGROUND OF THE INVENTION

Coronary artery disease, leading to myocardial infarction and ischemia, is currently a leading cause of morbidity and mortality worldwide. Current treatment alternatives consist of percutaneous transluminal angioplasty, stenting, and coronary artery bypass grafting (CABG). CABG can be carried out using either arterial or venous conduits and is the most effective and most widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. IH is characterized by phenotypic modulation, followed by de-adhesion and migration of medial and adventitial smooth muscle cells (SMCs) and myofibroblasts into the intima where they proliferate. In many cases, this response can lead to stenosis and diminished blood flow through the graft. It is thought that III may be initiated by the abrupt exposure of the veins to the dynamic mechanical environment of the arterial circulation.

### SUMMARY

For these and other reasons, there is a general need for systems, methods and devices that can provide enhanced AVGs and other improved grafts for mammalian patients. Desirably, the systems, methods, and devices may improve long term patency and minimize surgical and device complications such as those caused by improper or inadequate production of a graft device.

Embodiments of the systems and methods described herein can be directed to systems for producing graft devices for mammalian patients, as well as to methods for producing these graft devices.

According to an aspect of the present inventive concepts, a system for applying a polymer fiber matrix to a tubular conduit to create a graft device comprises a polymer solution including at least one polymer and at least one solvent. The system comprises a polymer delivery assembly constructed and arranged to receive the polymer solution and to deliver the polymer fiber matrix to the tubular conduit, a rotating assembly constructed and arranged to rotate at least one of the tubular conduit or the polymer delivery assembly, and a controller constructed and arranged to control the polymer delivery assembly and the rotating assembly. The system is constructed and arranged to reduce the amount of the solvent in the graft device.

In some embodiments, the reducing of the amount of solvent is configured to reduce an event selected from the group consisting of: non-favorable healing response of tissue; delayed healing response of tissue; loss of tissue viability; and combinations thereof.

In some embodiments, the system is configured to prevent or at least avoid implantation of the graft device during a minimum time period after delivery of the fiber matrix to the tubular conduit. The minimum time period can comprise a duration of approximately 10 minutes. The minimum time period can comprise a duration selected from the group consisting of: 2 minutes; 5 minutes; 7 minutes; and 10 minutes. The system can further comprise a preservative solution, the tubular conduit can comprise a blood vessel, and the blood vessel can be maintained in the preservative solution during the minimum time period. The system can further comprise a solvent-reducing element constructed and arranged to be activated during the minimum time period and to remove solvent from the graft device. The solvent-reducing element can comprise an element selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations thereof.

In some embodiments, the system further comprises a chamber surrounding the tubular conduit during delivery of the polymer fiber matrix to the tubular conduit. The chamber can be constructed and arranged to be disposed of after the creation of the graft device. The chamber can be constructed and arranged to extract solvent from the delivered polymer fiber matrix during and/or after application of the polymer fiber matrix to the tubular conduit. The chamber can further comprise a filter constructed and arranged to remove solvent from the chamber.

In some embodiments, the system further comprises a solvent-reducing element comprising one or more elements constructed and arranged to reduce the amount of solvent in the graft device. The solvent-reducing element can comprise one or more elements selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations thereof. The solvent-reducing element can comprise a fluid extraction element constructed and arranged to remove fluid containing the solvent from the chamber during and/or after application of the fiber matrix to the tubular conduit. The solvent-reducing element can comprise a temperature control element constructed and arranged to adjust temperature within the chamber to reduce the amount of solvent in the graft device. The solvent-reducing element can comprise a fluid delivery element constructed and arranged to deliver fluid into the chamber during and/or after application of the fiber matrix to the tubular conduit. The solvent-reducing element can further comprise a fluid sprayed by the fluid delivery element to a location proximate the tubular conduit and the fluid can be configured to enhance diffusion. The solvent-reducing element can comprise an agitating element. The agitating element can comprise a fan. The agitating element can be positioned proximate the tubular conduit. The agitating element can comprise a stream of at least one of laminar gas flow or turbulent gas flow proximate the tubular conduit. The solvent-reducing element can comprise a humidity control element. The solvent-reducing element can comprise at least a replaceable portion. The solvent-reducing element can be constructed and arranged to translate and/or rotate about the tubular conduit. The solvent-reducing element can be configured to translate and/or rotate relative to the tubular conduit.

In some embodiments, the system further comprises a solvent-reducing material. The system can be configured to apply the solvent-reducing material to the tubular conduit. The solvent-reducing material can comprise a solvent-absorbing material. The solvent-reducing material can comprise a material configured to chemically interact with the solvent. The solvent-reducing material can comprise a material selected from the group consisting of: desiccant; lipid; phospholipid; buffer; pH buffer; polyethylene; PTFE; fibrin; albumin; gelatin; oil; wax; PEG; carbon particle; activated carbon particle; alkaline material; powder; carbon particles; polymer beads; polymer gel; wicking fibrous membrane; solvent capillary transport system; ionizing gas; plasma; and combinations thereof. The solvent-reducing material can be configured as a barrier for preventing interaction of solvent in the fiber matrix with the tubular conduit. In some embodiments, the barrier material is further configured to absorb solvent. The solvent-reducing material can comprise a poloxamer gel. The solvent-reducing material can be constructed and arranged to be applied to at least one of the tubular conduit or the polymer fiber matrix. The solvent-reducing material can be constructed and arranged to be applied during application of the polymer fiber matrix to the tubular conduit. The solvent-reducing material can be constructed and arranged to be removed from at least one of the polymer fiber matrix or the tubular conduit prior to implantation of the graft device in a patient. The system can comprise a mandrel configured to be slidingly inserted into the tubular conduit, and the mandrel can be further configured to deliver the solvent-reducing material to the tubular conduit. The mandrel can comprise a porous mandrel. The system can comprise a modifying element configured to deliver the solvent-reducing material to the tubular conduit. The modifying element can comprise a nozzle. The polymer delivery assembly can be configured to deliver the solvent-reducing material to the tubular conduit.

In some embodiments, the system further comprises a sensor configured to produce a sensor signal, and the system can be configured to reduce the amount of solvent based on the sensor signal. The sensor can comprise multiple sensors, each configured to produce a sensor signal. The sensor can comprise one or more sensors selected from the group consisting of: optical sensor; temperature sensor; humidity sensor; pH sensor; ganged litmus paper instrument; strain gauge; accelerometer; load cell; electrochemical sensor; pressure sensor; chemical sensor; color changing chemical sensor; a photoionization sensor; and combinations thereof. The sensor can comprise a fluorine sensor. The sensor can comprise a temperature sensor constructed and arranged to measure cooling of the tubular conduit. The sensor can comprise a sensor constructed and arranged to measure the temperature between an inlet port and an outlet port of the chamber. The sensor can comprise a sensor constructed and arranged to measure a parameter selected from the group consisting of: weight of the graft device; mass of the graft device; acidity of the graft device; a parameter of the exhaust of a chamber surrounding the tubular conduit during delivery of the polymer fiber matrix to the tubular conduit; and combinations thereof. The system can be configured to adjust a system parameter based on the sensor signal. The adjusted system parameter can comprise a parameter selected from the group consisting of: rotational velocity of a mandrel within the tubular conduit; rotational velocity of the polymer delivery assembly; translation rate of the polymer delivery assembly; translation rate of a modification assembly; translation rate of the tubular conduit; flow rate of the polymer solution into the polymer delivery assembly; voltage applied between a nozzle and a mandrel inserted into the tubular conduit; an environmental parameter of a chamber surrounding the tubular conduit such as temperature within the chamber, humidity within the chamber, pressure within the chamber, temperature proximate the tubular conduit, humidity proximate the tubular conduit and/or pressure proximate the tubular conduit; flow rate of air or other gas into a chamber surrounding the tubular conduit; flow rate of air or other gas proximate the tubular conduit; delivery of a reducing agent (e.g. a solvent-reducing agent) onto or otherwise proximate the tubular conduit and/or the fiber matrix; distance between a nozzle and the tubular conduit; distance between a modification element and the tubular conduit; and combinations thereof. The system can be configured to adjust the system parameter at least one of prior to; during; or after delivery of the polymer fiber matrix to the tubular conduit. The sensor signal can represent a solvent parameter level, and the system can be configured to reduce the amount of solvent in the graft device until the solvent parameter level reaches a threshold. The reaching of the threshold can comprise the solvent parameter level falling below a maximum level. The system can be configured to perform a function until the solvent parameter reaches a threshold, and the function can be selected from the group consisting of: maintaining the graft device within a chamber of the system (e.g. via a locked door or other controlled-access point); rotating the graft device; providing a flow of gas proximate the graft device; providing an elevated temperature proximate the graft device; providing a desiccant proximate the graft device; and combinations thereof. The system can comprise a mandrel configured to be slidingly inserted into the tubular conduit, and the mandrel can comprise the sensor.

In some embodiments, the polymer delivery assembly is constructed and arranged to deliver at least one of hollow fibers or flat fibers to the tubular conduit. The polymer delivery assembly can be constructed and arranged to deliver fibers with an aspect ratio between 1.01:1 and 10:1.

In some embodiments, the system is constructed and arranged to reduce the amount of solvent in the graft device by rotating the tubular conduit. The system can be configured to rotate the tubular conduit during delivery of the polymer fiber matrix to the tubular conduit. The system can be configured to rotate the tubular conduit after the delivery of the polymer fiber matrix to the tubular conduit is complete. The system can be configured to reduce the amount of solvent in the graft device by rotating the tubular conduit at a rate above a threshold for at least 1 second (e.g. when the threshold equals the rotation rate present when the polymer fiber matrix is being delivered to the tubular conduit). The threshold can comprise a rotational speed of at least 250 rpm. The system can be configured to rotate the tubular conduit at a variable rate. The system can be configured to rotate the tubular conduit for a first rate during the delivery of the polymer fiber matrix to the tubular conduit, and at a second rate after the delivery of the polymer fiber matrix to the tubular conduit. The second rate can be greater than the first rate.

In some embodiments, the graft device is constructed and arranged as a bypass graft.

In some embodiments, the graft device is constructed and arranged as a coronary artery bypass graft.

In some embodiments, the graft device is constructed and arranged as a peripheral artery bypass graft.

In some embodiments, the graft device is constructed and arranged as at least one of: a neo-artery or a neo-vein.

In some embodiments, the graft device further comprises a kink resisting element. The kink resisting element can be positioned between the tubular conduit and the fiber matrix. The fiber matrix can comprise an inner layer and an outer layer, and the kink resisting element can be positioned between the fiber matrix inner layer and outer layer. The kink resisting element can be positioned outside of the fiber matrix. The kink resisting element can comprise a spine. The spine can comprise a first support portion and a second support portion, and at least one of the first support portion or the second support portion can be constructed and arranged to rotate relative to the other to receive the tubular conduit. The spine can comprise a first support portion comprising a first set of projections, and a second support portion comprising a second set of projections, and the first set of projections can interdigitate with the second set of projections. The kink resisting element can comprise at least one filament with a diameter between 0.4 mm and 0.5 mm. The kink resisting element can comprise a resiliently biased element. The kink resisting element can be resiliently biased with a heat treatment. The kink resisting element can comprise a surface treated element. The kink resisting element surface treatment can be configured to increase surface roughness of the kink resisting element.

In some embodiments, the polymer delivery assembly is configured to produce the fiber matrix with a thickness between approximately 220 μm and 280 μm.

In some embodiments, the polymer delivery assembly is configured to produce the fiber matrix with fibers comprising a diameter between 6 μm and 15 μm. The polymer delivery assembly can be configured to produce the fiber matrix with fibers comprising a diameter of approximately 7.8 μm. The polymer delivery assembly can be configured to produce the fiber matrix with fibers comprising a diameter of approximately 8.6 μm.

In some embodiments, the polymer delivery assembly is configured to produce the fiber matrix with a porosity between 40% and 80%. The polymer delivery assembly can be configured to produce the fiber matrix with a porosity of approximately 46.9%. The polymer delivery assembly can be configured to produce the fiber matrix with a porosity of approximately 50.4%.

In some embodiments, the polymer delivery assembly is configured to produce the fiber matrix with a compliance between approximately 0.2×10⁻⁴/mmHg and 3.0×10⁻⁴/mmHg.

In some embodiments, the polymer delivery assembly is configured to produce the fiber matrix with an elastic modulus between 10 MPa and 18 MPa.

In some embodiments, the polymer delivery assembly comprises at least one nozzle. The polymer delivery assembly can be configured to deliver polymer solution to the at least one nozzle at a flow rate between 10 ml/hr and 25 ml/hr. The at least one nozzle can comprise stainless steel. The polymer delivery assembly can further comprise a linear drive assembly configured to translate the at least one nozzle. The linear drive assembly can be configured to translate the nozzle at least 10 cm.

In some embodiments, the tubular conduit comprises harvested tissue. The harvested tissue can comprise tissue selected from the group consisting of: saphenous vein; vein; artery; urethra; intestine; esophagus; ureter; trachea; bronchi; duct; fallopian tube; and combinations thereof.

In some embodiments, the tubular conduit comprises artificial material. The artificial material can comprise a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations thereof.

In some embodiments, the polymer solution comprises a polymer weight to solvent volume ratio between 20% and 30%. The polymer solution can comprise a polymer weight to solvent volume ratio between 24% and 26%. The polymer solution can comprise a polymer weight to solvent volume ratio between 24.5% and 25.5%.

In some embodiments, the polymer solution comprises materials with molecular weight averages between 80,000 and 150,000.

In some embodiments, the polymer solution comprises a viscosity between 2000 cp and 24000 cp.

In some embodiments, the polymer solution comprises a conductivity between 0.4 μS/cm and 1.7 μS/cm.

In some embodiments, the polymer comprises a surface tension between 21.5 mN/m and 23.0 mN/m.

In some embodiments, the at least one polymer comprises at least two polymers. The at least one polymer can comprise a first polymer with a first hardness and a second polymer with a second hardness different than the first hardness. The first material can comprise polyhexamethylene oxide soft segments. The second material can comprise aromatic methylene diphenyl isocyanate hard segments.

In some embodiments, the at least one solvent comprises HFIP. The at least one solvent can comprise HFIP with a 99.97% minimum purity.

In some embodiments, the rotating assembly comprises at least one motor.

In some embodiments, the rotating assembly is configured to rotate at least one of the tubular conduit or the polymer delivery assembly at a rate between 100 rpm and 500 rpm. The rotating assembly can be configured to rotate at least one of the tubular conduit or the polymer delivery assembly at a rate between 200 rpm and 300 rpm. The rotating assembly can be configured to rotate at least one of the tubular conduit or the polymer delivery assembly at a rate between 240 rpm and 260 rpm.

In some embodiments, the rotating assembly is configured to rotate at least one of the tubular conduit or the polymer delivery assembly at a variable rate.

In some embodiments, the controller is configured to control a component selected from the group consisting of: the polymer delivery assembly; the rotating assembly; a linear drive assembly; a modification assembly; a voltage applied to a mandrel; and combinations thereof.

In some embodiments, the controller comprises an environmental controller. The environmental controller can be configured to remove solvent. The environmental controller can be configured to control one or more environmental parameters selected from the group consisting of: temperature; humidity; pressure; solvent concentration; and combinations thereof.

In some embodiments, the controller further comprises at least one gas propulsion mechanism. The at least one gas propulsion mechanism can comprise a fan.

In some embodiments, the controller is configured to detect an undesired state related to the solvent. The controller can comprise an alarm assembly. The alarm assembly can be configured to provide an alert selected from the group consisting of: audible alert; visual alert; tactile alert; and combinations thereof. The controller can be configured to stop delivery of the fiber matrix to the tubular conduit when an undesired state is detected.

According to another aspect of the present inventive concepts, a system for applying a polymer fiber matrix to a tubular conduit to create a graft device comprises a polymer delivery assembly constructed and arranged to receive a solution comprising polymer and solvent, and to deliver the polymer fiber matrix to the tubular conduit. The system can comprise a rotating assembly constructed and arranged to rotate at least one of the tubular conduit or the polymer delivery assembly, a controller constructed and arranged to control the polymer delivery assembly and the rotating assembly, and the system can be constructed and arranged to reduce solvent-caused adverse effects to the tubular conduit of the graft device.

In some embodiments, the tubular conduit comprises living tissue. The tubular conduit can comprise living tissue selected from the group consisting of: saphenous vein; vein; artery; urethra; intestine; esophagus; ureter; trachea; bronchi; duct; fallopian tube; and combinations thereof.

In some embodiments, the system further comprises a neutralizing agent configured to reduce the solvent-caused adverse effects. The system can be constructed and arranged to apply the neutralizing agent to the tubular conduit. The system can be constructed and arranged to apply the neutralizing agent at least one of: prior to; during; or after the delivery of the polymer fiber matrix to the tubular conduit. The neutralizing agent can comprise an agent selected from the group consisting of: a buffer; polyethylene; PTFE; fibrin; albumin; gelatin; PEG; carbon particle; activated carbon particle; sulfate; phosphate; ADP; ATP converted from ADP; an acid reducing material; a lipid; a phospholipid; an acidophilic bacteria; an alkaliphilic bacteria; and combinations thereof. The neutralizing agent can be configured as a barrier surrounding the tubular conduit to prevent interaction between the solvent and the tubular conduit. The barrier can be configured to be removed prior to implantation of the graft device in the patient. The barrier can comprise a material selected from the group consisting of: lipid; phospholipid; buffer; pH buffer; polyethylene; PTFE; fibrin; albumin; gelatin; oil; wax; PEG; carbon particle; activated carbon particle; alkaline material; powder; carbon particles; polymer beads; polymer gel; a poloxamer gel; and combinations thereof. The neutralizing agent can comprise a poloxamer gel. The system can comprise a mandrel configured to be slidingly inserted into the tubular conduit, and mandrel can be further configured to deliver the neutralizing agent to the tubular conduit. The mandrel can comprise a porous mandrel. The system can comprise a modifying element configured to deliver the neutralizing agent to the tubular conduit. The modifying element can comprise a nozzle. The polymer delivery assembly can be configured to deliver the neutralizing agent to the tubular conduit. In some embodiments, the neutralizing agent is further configured to absorb solvent.

According to another aspect of the present inventive concepts, a method of creating a graft device comprises using a system, as described herein, and causes the polymer delivery assembly of the system to deliver the polymer fiber matrix to the tubular conduit.

In some embodiments, the method further comprises implanting the graft device in the patient after a minimum time period has elapsed since the delivery of the polymer fiber matrix to the tubular conduit. The minimum time period can comprise a duration selected from the group consisting of: 2 minutes; 5 minutes; 7 minutes; and 10 minutes. The method can further comprise use of a preservative solution, the tubular conduit can comprise a blood vessel, and the blood vessel can be maintained in the preservative solution during the minimum time period.

In some embodiments, the tubular conduit comprises a vein segment, and the method further comprises placing the vein segment in preservative solution. The vein segment can be placed in the preservative solution prior to delivering the polymer fiber matrix to the tubular conduit. The method can further comprise placing the vein segment in the preservative solution after delivering the polymer fiber matrix to the tubular conduit. The preservative solution can comprise a material selected from the group consisting of: chilled fluid; fluid at approximately 4° C.; lactated ringers solution; papaverine; heparin; and combinations thereof.

### BRIEF DESCRIPTION OF THE DRAWINGS

The foregoing and other objects, features and advantages of embodiments of the technology described herein will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of a system for producing a graft device, consistent with the present inventive concepts.

FIG. 2 is a flow chart of a method for producing and implanting a graft device, consistent with the present inventive concepts.

FIG. 3, is a flow chart of a method for delivering polymer fibers to a tubular conduit while adjusting one or more system parameters, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

As used herein, the term “about” or “approximately” generally refers to the referenced numeric indication plus or minus 15% of that referenced numeric indication.

It will be understood that, when a range is recited, such as “between about X and about Y”, or “from about X to about Y”, that the range recited is inclusive of the end points X and Y.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.). A first component (e.g. a device, assembly, housing or other component) can be “attached”, “connected” or “coupled” to another component via a connecting filament (as defined below). In some embodiments, an assembly comprising multiple components connected by one or more connecting filaments is created during a manufacturing process (e.g. pre-connected at the time of an implantation procedure of the system of the present inventive concepts). Alternatively or additionally, a connecting filament can comprise one or more connectors (e.g. a connectorized filament comprising a connector on one or both ends), and a similar assembly can be created by a user (e.g. a clinician) operably attaching the one or more connectors of the connecting filament to one or more mating connectors of one or more components of the assembly.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

The terms “reduce”, “reducing”, “reduction” and the like, where used herein, are to include a reduction in a quantity, including a reduction to zero. Reducing the likelihood of an occurrence includes prevention of the occurrence.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable sub-combination. For example, it may be appreciated that all features set out in any of the claims (whether independent or dependent) can be combined in any given way.

Provided herein are systems and methods for producing graft devices for implantation in a mammalian patient, such as to carry fluids (e.g. blood or other body fluid) from a first anatomical location to a second anatomical location. The graft devices include a tubular conduit (e.g. a harvested blood vessel segment, other harvested tissue and/or an artificial conduit) and a fiber matrix (e.g. a polymer fiber matrix) that surrounds the tubular conduit. The systems can comprise a polymer delivery assembly configured to apply the fiber matrix about the tubular conduit. The systems described herein can include a solvent-reducing assembly for removing or at least reducing solvent from the graft device or the environment surrounding the graft device. Alternatively or additionally, the systems can be constructed and arranged to reduce adverse effects (e.g. reduce injury) to the tubular conduit caused by a solvent (e.g. HFIP) that comes into contact with the tubular conduit (e.g. a vein). Such adverse effects to be reduced include but are not limited to: non-favorable healing response of tissue; delayed healing response of tissue; and/or loss of tissue viability. Methods of reducing solvent present in the graft device (e.g. during or soon after the process of creating the device) as well as methods of reducing adverse effects of a solvent are also described.

The polymer delivery assembly can comprise one or more of: an electrospinning device; a melt-spinning device; a melt-electrospinning device; a misting assembly; a sprayer; an electrosprayer; a fuse deposition device; a selective laser sintering device; a three-dimensional printer; and/or other fiber matrix delivery device. The fiber matrix delivery process can be performed in an operating room, such as when the tubular conduit is a harvested saphenous vein segment to be anastomosed between the aorta and a location on a diseased coronary artery distal to an occlusion. In these cardiovascular bypass procedures, end to side anastomotic connections are typically used to attach the graft device to the aorta and the diseased artery. Alternatively, a side to side anastomosis can be used, such as to attach the graft device to multiple arteries in a serial fashion.

The fiber matrix can comprise one or more materials, such as one or more similar or dissimilar polymers as described in detail herebelow. The fiber matrix can comprise a biodegradable, bioerodible or bioabsorbable (hereinafter “biodegradable”) material or otherwise be configured such that the support to the graft device provided by the fiber matrix changes over time after implantation. Numerous biodegradable polymers can be used such as: polylactide, polyglycolide, polysaccharides, proteins, polyesters, polyhydroxyalkanoates, polyalkylene esters, polyamides, polycaprolactone, polyvinyl esters, polyamide esters, polyvinyl alcohols, polyanhydrides and their copolymers, modified derivatives of caprolactone polymers, poly trimethylene carbonate, polyacrylates, polyethylene glycol, hydrogels, photo-curable hydrogels, terminal diols, and combinations of these. Dunn et al. (U.S. Pat. No. 4,655,777) discloses a medical implant including bioabsorbable fibers that reinforce a bioabsorbable polymer matrix. Alternatively or additionally, the fiber matrix can comprise one or more portions including durable or otherwise relatively non-biodegradable materials, such as materials configured to remain intact within the patient's body for long periods of time when implanted, such as at least 6 months or at least 1 year.

The graft devices can further include one or more spines or other kink resisting elements (hereinafter “spine”) surrounding at least a portion of the tubular conduit, such as to prevent luminal narrowing, radial collapse, kinking and/or other undesired movement of the graft device (e.g. movement into an undesired geometric configuration), such as movement that occurs while implanting the graft device during a surgical procedure and/or at a time after implantation in the patient. One or more spines can be placed inside the tubular conduit, between the tubular conduit and the fiber matrix, between layers or within layers of the fiber matrix and/or outside the fiber matrix. The spine can comprise a biodegradable material or otherwise be configured to provide a temporary support to the graft device or a supporting force that otherwise reduces over time. Alternatively or additionally, the spine can comprise one or more portions including durable or otherwise non-biodegradable materials configured to remain intact for long periods of time when implanted, such as at least 6 months or at least 1 year. In some embodiments, the spine can be applied, placed and/or inserted about the tubular conduit by the fiber application assembly (e.g. automatically or semi-automatically via a robotic mechanism of the fiber application assembly) or with a placement insertion tool (e.g. manually by a clinician or other operator of the system).

The graft devices described herein can include an electrospun fiber matrix such as those disclosed in U.S. patent application Ser. No. 13/502,759, filed Apr. 19, 2012, the content of which is incorporated herein by reference in its entirety for all purposes. The technology described herein can include graft devices, as well as systems, tools and methods for producing and/or implanting graft devices, such as those disclosed in applicant's patents U.S. Pat. No. 8,992,594, filed Jun. 14, 2012, and U.S. Pat. No. 9,445,874, filed Jan. 18, 2013, the content of each of which is incorporated herein by reference in its entirety for all purposes. The technology described herein can include graft devices, as well as systems, tools and methods for producing and/or implanting graft devices, such as those disclosed in applicant's co-pending applications U.S. patent application Ser. No. 13/979,243, filed Jul. 11, 2013; U.S. patent application Ser. No. 13/984,249, filed Aug. 7, 2013; U.S. patent application Ser. No. 14/354,025, filed Apr. 24, 2014; U.S. patent application Ser. No. 14/378,263, filed Aug. 12, 2014; the content of each of which is incorporated herein by reference in its entirety for ail purposes.

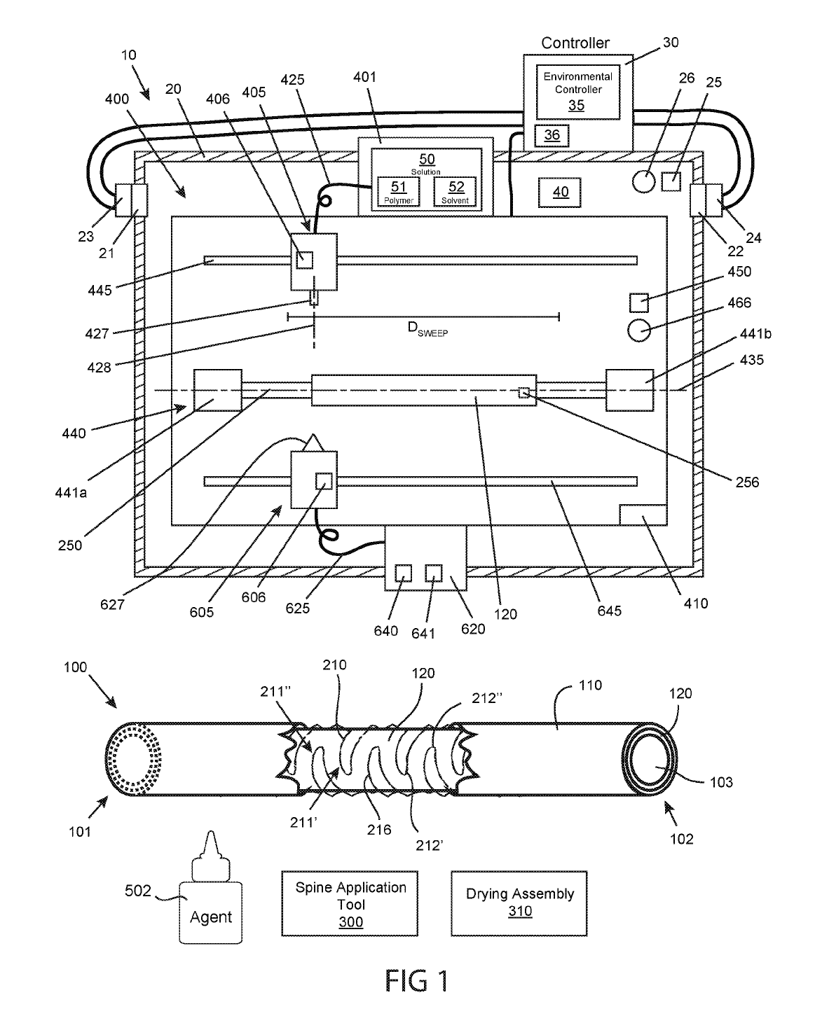

Referring now to FIG. 1, a schematic view of a system for producing a graft device comprising a tubular conduit with a surrounding fiber matrix is illustrated. System 10 includes various components and assemblies (hereinafter “components”) used to create a graft device for a mammalian patient, such as graft device 100 shown comprising a tubular conduit 120 surrounded by a fiber matrix 110. Graft device 100 can be constructed and arranged to perform as a bypass graft, such as a coronary artery bypass graft or a peripheral artery bypass graft. In some embodiments, graft device 100 is constructed and arranged as a neo-vessel, such as a neo-artery and/or a neo-vein. Tubular conduit 120 can include living tissue and/or artificial materials. In some embodiments, tubular conduit 120 comprises living tissue (e.g. harvested tissue), such as a segment or other portion of tissue selected from the group consisting of: saphenous vein; vein; artery; urethra; intestine; esophagus; ureter; trachea; bronchi; duct; fallopian tube; and combinations of one or more of these or other tissues. Alternatively or additionally, tubular conduit 120 can comprise an artificial material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations of one or more of these or other materials. Graft device 100 comprises first end 101, second end 102, and a lumen 103 extending from first end 101 to second end 102. System 10 includes fiber application assembly 400 configured to deliver the fiber matrix 110 about tubular conduit 120. Fiber application assembly 400 can comprise an electrospinning device. In some embodiments, fiber application assembly 400 comprises a device selected from the group consisting of: an electrospinning device; a melt-spinning device; a melt-electrospinning device; a misting assembly; a sprayer; an electrosprayer; a fuse deposition device; a selective laser sintering device; a three-dimensional printer; and combinations of one or more of these. System 10 can be configured to remove solvent 52, accelerate the removal of solvent 52 and/or at least reduce the amount of solvent 52 present in tubular conduit 120, fiber matrix 110, graft device 100, chamber 20 and/or another component of system 10 (hereinafter “remove solvent 52” or “remove solvent”). Alternatively or additionally, system 10 can be configured to reduce injury to the tubular conduit 120 by one or more solvents (e.g. reduce injury to living tissue such as living vein tissue). System 10 can include one or more sensors, such as sensors 26, 36, 256, 406, 466 and/or 606, shown in FIG. 1 and described in detail herebelow. Sensors 26, 36, 256, 406, 466 and/or 606 can provide a signal related to the presence of one or more solvents and/or a signal otherwise used to perform a solvent-reducing process and/or to reduce injury to a tubular conduit 120 by one or more solvents.

System 10 includes a mandrel 250 and fiber delivery assembly 400 comprises a rotating assembly 440 configured to rotate mandrel 250. In the embodiment shown in FIG. 1, mandrel 250 has been slidingly inserted into tubular conduit 120, and subsequently engaged with rotating assembly 440. Fiber delivery assembly 400 can include polymer solution dispenser 401 configured to dispense one or more polymer solutions, such as polymer solution 50 shown. Polymer solution 50 can include a mixture of one or more polymers 51, one or more solvents 52 and/or other materials present in and/or used to create fiber matrix 110. Polymer solution 50 can comprise a cartridge or other reservoir surrounding polymers 51 and solvents 52, the reservoir being fluidly attachable to polymer solution dispenser 401 by an operator of system 10.

System 10 can include an environmentally controllable chamber, chamber 20 shown, which surrounds at least a portion of mandrel 250 when mandrel 250 is operably attached to fiber delivery assembly 400 (e.g. chamber 20 surrounds at least tubular conduit 120 and fiber matrix 110 during the creation of graft device 100). Chamber 20 can further surround one or more portions of polymer delivery assembly 405 and/or modification assembly 605 described herebelow. In some embodiments, chamber 20 comprises a disposable cartridge and/or at least a portion of chamber 20 is disposable (e.g. used to make one or more graft devices 100 for a single patient in a single clinical procedure). Chamber 20 can be configured to remove solvent 52 during and/or after application of fiber matrix 110 to tubular conduit 120 (e.g. removal of solvent 52 during a pre-determined wait period that occurs after completion of delivery of fiber matrix 110).

System 10 includes controller 30 which is configured to provide control signals and/or receive information signals. Controller 30 can be configured to control one or more of: polymer delivery assembly 405 (e.g. to control the flow rate of polymer solution 50); rotating assembly 440 (e.g. to control the rotation of mandrel 250); linear drive assembly 445 (e.g. to control the translation rate or position of polymer delivery assembly 405); modification assembly 605 (e.g. to control delivery of material by modification assembly 605, delivery of energy by modification assembly 605 and/or removal of solvent 52 by modification assembly 605); linear drive assembly 645 (e.g. to control the translation rate or position of modification assembly 605); voltage applied to mandrel 250 (e.g. voltage provided by power supply 410); and combinations of one or more of these. Controller 30 can further comprise environmental controller 35. Environmental controller 35 can be configured to remove solvent 52. Alternatively or additionally, environmental controller 35 can be configured to control an environmental parameter within chamber 20, such as an environmental parameter selected from the group consisting of: temperature; humidity; pressure; solvent 52 concentration; and combinations of one or more of these. Environmental controller 35 or another component of controller 30 can comprise one or more fans or other gas propulsion mechanisms, such as to provide air or other gas to inlet port 21 (e.g. via the tube shown positioned between controller 30 and inlet port 21) or extract gas from chamber 20 via outlet port 22 (e.g. via the tube shown positioned between controller 30 and outlet port 22). In some embodiments, controller 30 comprises an alarm assembly, which can be constructed and arranged to be activated when an undesired state is detected (e.g. an undesired concentration or amount of solvent 52 present, or other undesired state related to solvent 52), such as to notify an operator of system 10. Controller 30 can comprise an alarm assembly constructed and arranged to provide an alert selected from the group consisting of: audible alert; visual alert; tactile alert; and combinations of one or more of these alerts. In some embodiments, when an undesired state is detected (e.g. an unacceptable concentration of solvent 52 within chamber 20, within tubular conduit 120 and/or within fiber matrix 110 is detected), application of fiber matrix 110 to tubular conduit 120 is stopped. Alternatively or additionally, after detection of the undesired state is detected, one or more parameters of system can be adjusted and the processed continued or re-started.

In some embodiments, system 10 comprises one or more similar or dissimilar spines 210, and graft device 100 comprises one or more of the spines 210. System 10 can include spine application tool 300, which can comprise a manual or automated (e.g. robotic) tool used to place spine 210 about tubular conduit 120, such as between one or more layers of fiber matrix 110 (e.g. between an inner layer with a first thickness, and an outer layer with a second thickness approximately twice as thick as the first layer's thickness). In some embodiments, system 10 can include one or more tools, components, assemblies and/or otherwise be constructed and arranged as described in applicant's co-pending U.S. patent application Ser. No. 15/023,265, filed Mar. 18, 2016, the content of which is incorporated herein by reference in its entirety for all purposes.

Mandrel 250 can comprise a metal mandrel, such as a mandrel constructed of 304 or 316 series stainless steel. Mandrel 250 can comprise a mirror-like surface finish, such as a surface finish with an Rₐ of approximately 0.1 μm to 0.8 μm. In some cases, Rₐ may be about 0.1 μm. In some cases, Rₐ may be about 0.2 μm. In some cases, Rₐ may be about 0.3 μm. In some cases, Rₐ may be about 0.4 μm. In some cases, Rₐ may be about 0.5 μm. In some cases, Rₐ may be about 0.6 μm. In some cases, Rₐ may be about 0.7 μm. In some cases, Rₐ may be about 0.8 μm. In some cases, Rₐ may be from about 0.1 μm to about 0.5 μm. In some cases, Rₐ may be from about 0.3 μm to about 0.8 μm. Mandrel 250 can comprise a length of up to about 45 cm, such as a length of between about 30 cm and about 45 cm, or between about 38 cm and about 40 cm. In some cases, a mandrel length may be from about 30 cm to about 45 cm. In some cases, a mandrel length may be about 25 cm. In some cases, a mandrel length may be about 30 cm. In some cases, a mandrel length may be about 35 cm. In some cases, a mandrel length may be about 40 cm. In some cases, a mandrel length may be about 45 cm. In some cases, a mandrel length may be about 50 cm. In some embodiments, system 10 includes multiple mandrels 250 with multiple different geometries, such as a set of mandrels 250 with different diameters (e.g. diameters of about 3.0 mm, about 3.5 mm, about 4.0 mm, and/or about 4.5 mm). In some cases, a set of mandrels may comprise at least 2 mandrels. In some cases, a set of mandrels may comprise at least 3 mandrels. In some cases, a set of mandrels may comprise 2 mandrels. In some cases, a set of mandrels may comprise 3 mandrels. A mandrel may comprise a diameter of about 3.0 mm. A mandrel may comprise a diameter of about 3.5 mm. A mandrel may comprise a diameter of about 4.0 mm. A mandrel may comprise a diameter from about 2.5 mm to about 5.0 mm. A first mandrel and a second mandrel of a set of mandrels may comprise different diameters. For example, a first mandrel may comprise a diameter of about 3.0 mm and a second mandrel may comprise a diameter of about 4.0 mm. A first mandrel and a second mandrel of a set of mandrels may comprise same diameters. For example, a first mandrel may comprise a diameter of about 3.0 mm and a second mandrel may comprise a diameter of about 3.0 mm. In some embodiments, fiber application assembly 400 is configured to automatically detect the mandrel 250 diameter (e.g. and to adjust rotation rate and/or another system parameter based on the detected mandrel 250 diameter). Each end of mandrel 250 is inserted into driving elements of rotating assembly 440, motors 441a and 441b, respectively, such that mandrel 250 can be rotated about axis 435 during application of fiber matrix 110. In some embodiments, a single motor drives one end of mandrel 250, with the opposite end attached to a rotatable attachment element (e.g. a bearing) of fiber application assembly 400.

Mandrel 250 can comprise a porous mandrel, such as a mandrel configured to deliver one or more drugs or other agents to tubular conduit 120 prior to, during and/or after application of fiber matrix 110 to tubular conduit 120. In some embodiments, an agent 502 is delivered to (e.g. coated onto) tubular conduit 120 via a porous mandrel 250, via polymer delivery assembly 405 (e.g. via nozzle 427), via modification assembly 605 (e.g. via modifying element 627), or otherwise. Agent 502 can comprise a solvent-reducing material (e.g. a material configured to absorb solvent and a material configured as a barrier that prevents solvent from reaching tubular conduit 120), a solvent neutralizing material, a hydrating solution and/or a preservative solution. In some embodiments, agent 502 comprises a preservative solution comprising one or more materials selected from the group consisting of: chilled fluid; fluid chilled to approximately 4° C.; water; saline; heparin; heparinized saline; blood; ringers solution; and combinations of one or more of these. In some embodiments, agent 502 comprises a material configured as both a barrier and a solvent-absorbing material.

Fiber application assembly 400 can include one or more polymer delivery assemblies, and in the illustrated embodiment, fiber application assembly 400 includes polymer delivery assembly 405. Polymer delivery assembly 405 comprises nozzle 427. Nozzle 427 includes an orifice constructed and arranged to deliver fiber matrix 110 to tubular conduit 120. Nozzle 427 can be a tubular structure including nozzle central axis 428. Nozzle 427 can be constructed of stainless steel, such as passivated 304 stainless steel. In some embodiments, nozzle 427 comprises an outer tube and an inner tube, such as to avoid icicle formation about nozzle 427. For example, polymer delivery assembly 405 and/or nozzle 427 can be constructed and arranged as described in applicant's co-pending application U.S. patent application Ser. No. 15/036,304, filed May 12, 2016.

Polymer delivery assembly 405 is fluidly attached to polymer solution dispenser 401 via delivery tube 425. Polymer delivery assembly 405 receives polymer solution 50 and delivers polymer fibers to tubular conduit 120, for example via an electrospinning process in which a voltage is applied between mandrel 250 and nozzle 427. Polymer delivery assembly 405 can comprise one or more pumping mechanisms, such as a syringe pump (e.g. a syringe pump in which polymer solution 50 is contained within the syringe), a peristaltic pump, a displacement pump and/or other pumping mechanism. Polymer delivery assembly 405 can further comprise linear drive assembly 445. Linear drive assembly 445 translates nozzle 427 in at least one direction for a linear travel distance D_{SWEEP} as shown. In some embodiments, linear drive assembly 445 reciprocally translates nozzle 427 along the distance D_{SWEEP}. In some embodiments, D_{SWEEP} comprises a length of approximately 30 cm, such as a length of at least 10 cm, 20 cm, 30 cm, 35 cm, or 40 cm.

As described hereabove, mandrel 250 can be rotated about axis 435 during the delivery of polymer fibers to tubular conduit 120 by polymer delivery assembly 405. Alternatively or additionally, polymer delivery assembly 405 (e.g. and nozzle 427) can rotate about mandrel 250 during delivery of the polymer fibers, e.g. as polymer delivery assembly 405 and mandrel 250 translate relative to each other (e.g. via translational motion of either or both).

In some embodiments, polymer solution 50 comprises two or more polymers 51, such as a first polymer 51a with a first hardness, and a second polymer 51b with a second hardness different than the first hardness. Polymer solution 50 can comprise a mixture of similar or dissimilar amounts of polyhexamethylene oxide soft segments, and aromatic methylene diphenyl isocyanate hard segments. Polymer solution 50 can further comprise one or more solvents 52, such as HFIP (e.g. HFIP with a 99.97% minimum purity). Polymer solution 50 can comprise one or more polymers 51 in a concentrated solution fully or at least partially solubilized within a solvent 52 and comprise a polymer weight to solvent volume ratio between about 20% and about 35%, such as a concentration between about 24% and about 26%, or between about 24.5% and about 25.5%. A polymer weight to solvent volume ratio may be about 20%. A polymer weight to solvent volume ratio may be about 25%. A polymer weight to solvent volume ratio may be about 30%. A polymer weight to solvent volume ratio may be about 35%. A polymer weight to solvent volume ratio may be about: 20%, 21%, 22%, 23%, 24%, 25%, 26%, 27%, 28%, 29%, 30%, 31%, 32%, 33%, 34%, or 35%.

Polymer solution 50 can comprise one or more materials with a molecular weight average (Mw) between about 80,000 and about 150,000 (PDI−Mw/Mn=2.1−3.5). A molecular weight average of the one or more materials may be from about 50,000 to about 250,000. A molecular weight average of the one or more materials may be from about 100,000 to about 150,000. A molecular weight average of the one or more materials may be from about 80,000 to about 150,000. A molecular weight average of the one or more materials may be from about 100,000 to about 250,000. A molecular weight average of the one or more materials may be at least about 80,000. A molecular weight average of the one or more materials may be at least about 90,000. A molecular weight average of the one or more materials may be at least about 100,000. A molecular weight average of the one or more materials may be less than about 110,000. A molecular weight average of the one or more materials may be less than about 120,000. A molecular weight average of the one or more materials may be less than about 130,000. A molecular weight average of the one or more materials may be less than about 140,000. A molecular weight average of the one or more materials may be less than about 150,000.

Polymer solution 50 can comprise a solution with a viscosity between about 2000 cP and about 2400 cP (e.g. measured at 25° C. and with shear rate=20 s⁻¹). A viscosity of a solution may be from about 1000 cP to about 3000 cP. A viscosity of a solution may be from about 1500 cP to about 3000 cP. A viscosity of a solution may be from about 2000 cP to about 3000 cP. A viscosity of a solution may be from about 1800 cP to about 2600 cP. A viscosity of a solution may be from about 2100 cP to about 2300 cP.

Polymer solution 50 can comprise a solution with a conductivity between about 0.4 μS/cm and about 1.7 μS/cm (e.g. measured at a temperature between 20° C. and 22° C.). A conductivity of a solution may be from about 0.1 μS/cm to about 3.0 μS/cm. A conductivity of a solution may be from about 0.1 μS/cm to about 2.5 μS/cm. A conductivity of a solution may be from about 0.1 μS/cm to about 2.0 μS/cm. A conductivity of a solution may be from about 0.1 μS/cm to about 1.5 μS/cm. A conductivity of a solution may be from about 0.5 μS/cm to about 1.5 μS/cm. A conductivity of a solution may be from about 0.5 μS/cm to about 2.0 μS/cm.

Polymer solution 50 can comprise a solution with a surface tension between about 21.5 mN/m and about 23.0 mN/m (e.g. measured at 25° C.). A surface tension of a solution may be from about 20 mN/m to about 25 mM/m. A surface tension of a solution may be from about 15 mN/m to about 25 mM/m. A surface tension of a solution may be from about 20 mN/m to about 23 mM/m. A surface tension of a solution may be from about 21 mN/m to about 25 mM/m. A surface tension of a solution may be from about 21 mN/m to about 22 mM/m. A surface tension of a solution may be from about 20 mN/m to about 24 mM/m. A surface tension of a solution may be from about 15 mN/m to about 23 mM/m.

In some embodiments, system 10 is constructed and arranged to produce a fiber matrix 110 with a thickness (e.g. absent of any spine 210) of between approximately 220 μm and 280 μm. A thickness of a fiber matrix may be from about 200 μm to about 300 μm. A thickness of a fiber matrix may be from about 150 μm to about 350 μm. A thickness of a fiber matrix may be from about 215 μm to about 300 μm. A thickness of a fiber matrix may be from about 220 μm to about 250 μm. A thickness of a fiber matrix may be from about 250 μm to about 280 μm. A thickness of a fiber matrix may be from about 200 μm to about 230 μm. A thickness of a fiber matrix may be from about 2700 μm to about 300 μm. Fiber matrix 110 can comprise a matrix of fibers with a diameter between 6 μm and 15 μm, such as a matrix of fibers with an average diameter between 7.8 μm and 8.6 μm, with an average diameter of approximately 7.8 μm, or with an average diameter of approximately 8.6 μm. A diameter of a matrix of fibers may be from about 5 μm to about 20 μm. A diameter of a matrix of fibers may be from about 6 μm to about 10 μm. A diameter of a matrix of fibers may be from about 10 μm to about 15 μm. A diameter of a matrix of fibers may be from about 6 μm to about 10 μm. A diameter of a matrix of fibers may be from about 7 μm to about 9 μm. An average diameter of a matrix of fibers may be about from about 7 μm to about 9 μm. An average diameter of a matrix of fibers may be about from about 6 μm to about 8 μm. Fiber matrix 110 can comprise a porosity of between about 40% and about 80%, such as a fiber matrix with a porosity between about 46.9% and about 50.4%, or with an average porosity of about 50.4% or about 46.9%. A porosity of a fiber matrix may be from about 45% to about 75%. A porosity of a fiber matrix may be from about 50% to about 70%. A porosity of a fiber matrix may be from about 45% to about 65%. A porosity of a fiber matrix may be from about 45% to about 60%. A porosity of a fiber matrix may be at least about 45%. A porosity of a fiber matrix may be at least about 40%. An average porosity of a fiber matrix may be from about 45% to about 60%. An average porosity of a fiber matrix may be from about 45% to about 55%. An average porosity of a fiber matrix may be from about 45% to about 50%. In some embodiments, fiber matrix 110 comprises an average compliance (“compliance” herein) between approximately 0.2×10⁻⁴/mmHg and approximately 3.0×10⁻⁴/mmHg, such as when measured in common arterial pressure ranges (e.g. arterial pressure ranges in healthy individuals and/or patients with cardiovascular disease). An average compliance of a fiber matrix may be from about 0.1×10⁻⁴/mmHg to about 4.0×10⁻⁴/mmHg. An average compliance of a fiber matrix may be from about 0.1×10⁻⁴/mmHg to about 3.0×10⁻⁴/mmHg. An average compliance of a fiber matrix may be from about 0.1×10⁻⁴/mmHg to about 2.0×10⁻⁴/mmHg. An average compliance of a fiber matrix may be from about 0.1×10⁻⁴/mmHg to about 1.0×10⁻⁴/mmHg. An average compliance of a fiber matrix may be from about 0.5×10⁻⁴/mmHg to about 4.0×10⁻⁴/mmHg. An average compliance of a fiber matrix may be from about 1×10⁻⁴/mmHg to about 4.0×10⁻⁴/mmHg. In some embodiments, fiber matrix 110 comprises an elastic modulus between about 10 MPa and about 18 MPa. A fiber matrix may comprise an elastic modulus of about 10 MPa. A fiber matrix may comprise an elastic modulus of about 15 MPa. A fiber matrix may comprise an elastic modulus of about 20 MPa. A fiber matrix may comprise an elastic modulus from about 5 MPa to about 20 MPa. A fiber matrix may comprise an elastic modulus from about 10 MPa to about 15 MPa. A fiber matrix may comprise an elastic modulus from about 15 MPa to about 20 MPa. A fiber matrix may comprise an elastic modulus from about 12 MPa to about 16 MPa.

Polymer delivery assembly 405 can be configured to deliver polymer solution 50 to nozzle 427 at a flow rate of between about 10 milliliters per hour (ml/hr) and about 25 ml/hr, such as at a flow rate between about 15 ml/hr and about 20 ml/hr, or a flow rate of approximately 15 ml/hr or approximately 20 ml/hr. In some embodiments, polymer delivery assembly 405 delivers polymer solution 50 to nozzle 427 at a flow rate of at least about 10 ml/hr. A flow rate may be from about 5 ml/hr to about 30 ml/hr. A flow rate may be from about 5 ml/hr to about 25 ml/hr. A flow rate may be from about 10 ml/hr to about 30 ml/hr. A flow rate may be from about 10 ml/hr to about 25 ml/hr. A flow rate may be from about 10 ml/hr to about 20 ml/hr. A flow rate may be from about 12 ml/hr to about 18 ml/hr.

As described above, in some embodiments, system 10 is constructed and arranged to produce a graft device 100 including a spine 210. Spine 210 can comprise multiple spines 210 with different inner diameters (IDs), such as multiple spines with IDs of approximately: 4.0 mm, 4.7 mm, and/or 5.5 mm. An inner diameter of a spine may be from about 3.5 mm to about 6.0 mm. An inner diameter of a spine may be from about 4.0 mm to about 5.5 mm. An inner diameter of a spine may be from about 4.0 mm to about 5.0 mm. An inner diameter of a spine may be from about 4.5 mm to about 5.0 mm. A first spine may comprise a different diameter than a second spine. For example, a first spine may comprise an inner diameter of about 4.0 mm and a second spine may comprise an inner diameter of about 5.5 mm. A first spine may comprise a same diameter as a second spine. For example, a first spine may comprise an inner diameter of about 4.0 mm and a second spine may comprise an inner diameter of about 4.0 mm. Spine 210 can comprise a filament 216 with a diameter of approximately 0.4 mm (e.g. for a spine with an ID between 4.0 mm and 4.7 mm). Spine 210 can comprise a filament 216 with a diameter of approximately 0.5 mm (e.g. for a spine with an ID between 4.8 mm and 5.5 mm). A diameter of a filament may be from about 0.1 mm to about 1.0 mm. A diameter of a filament may be from about 0.2 mm to about 0.8 mm. A diameter of a filament may be from about 0.3 mm to about 0.7 mm. A diameter of a filament may be from about 0.4 mm to about 0.6 mm. Spine 210 can comprise a series of inter-digitating fingers spaced approximately 0.125 inches from each other so that the recurring unit of spine including one left finger and one right finger occurs every 0.25 inches. A series of inter-digitating fingers may be spaced about 0.1 inches from each other. A series of inter-digitating fingers may be spaced about 0.125 inches from each other. A series of inter-digitating fingers may be spaced about 0.150 inches from each other. A series of inter-digitating fingers may be spaced about 0.175 inches from each other. A series of inter-digitating fingers may be spaced about 0.2 inches from each other. A series of inter-digitating fingers may be spaced from about 0.05 inches to about 0.2 inches from each other. This recurring feature length (i.e. recurring unit of spine) can have a range comprised between about 0.125 inches and about 0.375 inches. The fingers can overlap in a symmetric and/or asymmetric pattern, such as an overlap of opposing fingers between about 2.5 mm and about 1.0 mm around the circumferential perimeter of spine 210. Spine 210 can be heat treated to achieve a resilient bias. Spine 210 can be surface-treated (e.g. with dimethylformamide) to increase the surface roughness and reduce crystallinity (e.g. to improve solvent-based adhesion with the deposited electrospun material, fiber matrix 110).

Spine 210 can include one or more portions that are resiliently biased, such as a resilient bias configured to provide a radial outward force at locations proximate ends 101 and/or 102, such as to provide a radial outward force to support or enhance the creation of an anastomosis during a cardiovascular bypass procedure. In some embodiments, spine 210 includes one or more portions that are malleable.

Spine 210 can include multiple curved projections 211′ and 211″, collectively 211. Projections 211′ each include a tip portion 212′ and projections 211″ each include a tip portion 212″ (singly or collectively, tip portions 212). Tip portions 212 can be arranged in the overlapping arrangement shown in FIG. 1. Projections 211′ and 211″ can comprise a first and second support portion, respectively, that are arranged such that at least one rotates relative to the other to create an opening to receive tubular conduit 120. In some embodiments, each tip portion 212 can comprise a diameter between about 0.020 inches and about 0.064 inches, such as a diameter approximating 0.042 inches. A tip portion may comprise a diameter from about 0.01 inches to about 0.1 inches. A tip portion may comprise a diameter from about 0.01 inches to about 0.08 inches. A tip portion may comprise a diameter from about 0.02 inches to about 0.05 inches. A tip portion may comprise a diameter from about 0.04 inches to about 0.08 inches. Projections 211 can each comprise a loop of a filament (e.g. a loop of a continuous filament), and projections 211′ and 211″ can be arranged in an interdigitating arrangement such as the alternating, interdigitating arrangement shown in FIG. 1. In some embodiments, the interdigitating projections 211′ and 211″ can overlap (e.g. spine 210 covers more than 360° of conduit 120). In some embodiments, projections 211′ and 211″ are arranged with an overlap of at least about 1.0 mm, at least about 1.1 mm, at least about 1.2 mm, at least about 1.3 mm, at least about 1.4 mm, at least about 1.5 mm, or at least about 1.6 mm. In some embodiments, spine 210 can be constructed and arranged as described in applicant's co-pending U.S. patent application Ser. No. 15/023,265, filed Mar. 18, 2016, the content of which is incorporated herein by reference in its entirety for all purposes.

Spine 210 can comprise at least three projections 211, such as at least six projections 211. In some embodiments, spine 210 includes at least two projections 211 for every about 15 mm of length of spine 210, such as at least two projections 211 for every about 7.5 mm of length of spine 210, or at least two projections for every about 2 mm of length of spine 210. In some cases, a spine may include at least two projections for every about 20 mm of length of spine. In some cases, a spine may include at least two projections for every about 10 mm of length of spine. In some cases, a spine may include at least two projections for every about 8 mm of length of spine. In some cases, a spine may include at least two projections for every about 6 mm of length of spine. In some cases, a spine may include at least two projections for every about 4 mm of length of spine. In some cases, a spine may include at least two projections for every about 1 mm of length of spine. In some embodiments, spine 210 comprises two projections 211 for each approximately 6.5 mm of length of spine 210. In some embodiments, a series of projections 211 are positioned approximately 0.125 inches from each other.

Spine 210 can comprise one or more continuous filaments 216, such as three or less continuous filaments, two or less continuous filaments, or a single continuous filament. In some embodiments, spine 210 comprises a continuous filament 216 of at least about 15 inches long (i.e. the curvilinear length), or at least about 30 inches long, such as when spine 210 comprises a length of approximately 3.5 inches. In some embodiments, filament 216 comprises a length (e.g. a continuous curvilinear length or a sum of segments with a cumulative curvilinear length) of approximately 65 inches (e.g. to create a 4.0 mm diameter spine 210), or a length of approximately 75 inches (e.g. to create a 4.7 mm diameter spine 210), or a length of approximately 85 inches (e.g. to create a 5.5 mm diameter spine 210). Filament 216 can comprise a relatively continuous cross section, such as an extruded or molded filament with a relatively continuous cross section. Spine 210 can comprise a filament 216 including at least a portion with a cross sectional geometry selected from the group consisting of: elliptical; circular; oval; square; rectangular; trapezoidal; parallelogram-shaped; rhomboid-shaped; T-shaped; star-shaped; spiral-shaped; (e.g. a filament comprising a rolled sheet); and combinations of one or more of these or other geometries. Filament 216 can comprise a cross section with a major axis between approximately 0.2 mm and approximately 1.5 mm in length, such as a circle or oval with a major axis less than or equal to about 1.5 mm, less than or equal to about 0.8 mm, or less than or equal to about 0.6 mm, or between about 0.4 mm and about 0.5 mm. Filament 216 can comprise a cross section with a major axis greater than or equal to about 0.1 mm, such as a major axis greater than or equal to about 0.3 mm. In some embodiments, the major axis and/or cross sectional area of filament 216 is proportionally based to the diameter of spine 210 (e.g. a larger spine 210 diameter correlates to a larger filament 216 diameter, such as when a range of different diameter spine 210's are provided in a kit).

Filament 216 can be a single core, monofilament structure. Alternatively, filament 216 can comprise multiple filaments, such as a braided multiple filament structure. In some embodiments, filament 216 can comprise an injection molded component or a thermoset plastic component, such as when spine 210 comprises multiple projections 211 that are created at the same time as the creation of one or more filaments 216 (e.g. when filament 216 is created in a three dimensional biased shape).

Filament 216 can comprise an electrospun component, such as a component produced by the same electrospinning device used to create fiber matrix 110, such as when spine 210 and fiber matrix 110 comprise the same or similar materials.

Spine 210 can comprise a material with a durometer between 52D and 120R, such as between 52D and 85D, such as between 52D and 62D. In some embodiments, spine 210 comprises a material with a durometer of approximately 55D. Spine 210 can comprise one or more polymers, such as a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluronic acid; silk fibroin collagen; elastin; poly(p-dioxanone); poly(3-hydroxybutyrate); poly(3-hydroxyvalerate); poly(valecrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of one or more of these or other materials.

Spine 210 can comprise the same or substantially similar material(s) as fiber matrix 110. Spine 210 can comprise at least one thermoplastic co-polymer. Spine 210 can comprise two or more materials, such as a first material and a second material harder than the first material. In some embodiments, spine 210 comprises relatively equal amounts of a harder material and a softer material. The softer material can comprise polydimethylsiloxane and a polyether-based polyurethane, and the harder material can comprise aromatic methylene diphenyl isocyanate. Spine 210 can comprise one or more drugs or other agents, such as one or more agents constructed and arranged to be released over time.

In some embodiments, spine 210 comprises a metal material, such as a metal selected from the group consisting of: a nickel titanium alloy; a titanium alloy; titanium; stainless steel; tantalum; magnesium; cobalt-chromium alloy; gold; platinum; and combinations of one or more of these or other materials. In some embodiments, spine 210 comprises a reinforced resin, such as a resin reinforced with carbon fiber and/or Kevlar. In some embodiments, at least a portion of spine 210 is biodegradable, such as when spine 210 comprises a biodegradable material such as a biodegradable metal or biodegradable polymer. In these embodiments, fiber matrix 110 can further comprise a non-biodegradable material. In some embodiments, spine 210 does not comprise a biodegradable material.

Spine 210 can be configured to biodegrade over time such as to provide a temporary kink resistance or other function to graft device 100. In one embodiment, spine 210 can temporarily provide kink resistance to graft device 100 for a period of less than twenty-four hours. In an alternative embodiment, spine 210 can provide kink resistance to graft device 100 for a period of less than one month. In yet another embodiment, spine 210 can provide kink resistance to graft device 100 for a period of less than six months. Numerous forms of biodegradable materials can be employed. Bolz et al. (U.S. Pat. No. 6,287,332) discloses a bioabsorbable implant which includes a combination of metal materials that can be an alloy or a local galvanic element. Metal alloys can consist of at least a first component which forms a protecting passivation coat and a second component configured to ensure sufficient corrosion of the alloy. The first component is at least one component selected from the group consisting of: magnesium, titanium, zirconium, niobium, tantalum, zinc and silicon, and the second component is at least one metal selected from the group consisting of: lithium, sodium, potassium, manganese, calcium and iron. Furst et al. (U.S. patent application Ser. No. 11/368,298) discloses an implantable device at least partially formed of a bioabsorbable metal alloy that includes a majority weight percent of magnesium and at least one metal selected from calcium, a rare earth metal, yttrium, zinc and/or zirconium. Doty et al. (U.S. patent application Ser. No. 11/744,977) discloses a bioabsorbable magnesium reinforced polymer stent that includes magnesium or magnesium alloys. Numerous biodegradable polymers can be used such as are described hereabove.

System 10 can include drying assembly 310, which is constructed and arranged to remove moisture from tubular conduit 120 and/or fiber matrix 110, such as to remove solvent 52 from locations surrounding tubular conduit 120 and/or fiber matrix 110. Drying assembly 310 can comprise a heat generator, dehydrator, desiccant or other fluid absorbing material, and/or other mechanism configured to remove solvent 52 from locations on, within, and/or proximate tubular conduit 120 and/or fiber matrix 110. Drying assembly 310 can comprise a handheld device. In some embodiments, tubular conduit 120 comprises harvested tissue (e.g. a harvested saphenous vein segment) and drying assembly 310 comprises gauze or other material used to manually remove fluids from tubular conduit 120, such as to remove solvents and/or improve adherence between fiber matrix 110 and tubular conduit 120.

Fiber application assembly 400 can include one or more modification assemblies constructed and arranged to modify one or more components and/or one or more portions of graft device 100. In the illustrated embodiment, fiber application assembly 400 includes modification assembly 605. Modification assembly 605 comprises a nozzle assembly or other modifying element, modifying element 627. Modification assembly 605 further comprises linear drive assembly 645. Assembly 605 is operably attached to linear drive assembly 645, which is configured to translate assembly 605 in at least one direction, such as in a reciprocating motion including back and forth motions spanning a distance similar to D_{SWEEP} of linear drive assembly 445. Assembly 605 can be operably attached to supply 620 via delivery tube 625.

Modification assembly 605 can be configured to remove vapor from one or more locations near to tubular conduit 120 and/or fiber matrix 110, such as to reduce the amount of solvent in tubular conduit 120 and/or fiber matrix 110. In these embodiments, supply 620 can comprise a vacuum that enables modification element 627 (e.g. a nozzle) to extract gas and/or vapor from these locations via delivery tube 625.

System 10 can include one or more graft device 100 modifying agents, such as agent 502 shown. Agent 502 can comprise a solvent or other agent configured to perform a surface modification, such as a solvent selected from the group consisting of: dimethylformamide; hexafluoroisopropanol; tetrahydrofuran; dimethyl sulfoxide; isopropyl alcohol; ethanol; and combinations of one or more of these or other solvents. In some embodiments, system 10 is constructed and arranged to perform a surface modification configured to enhance the adhesion of two or more of tubular conduit 120, spine 210 and fiber matrix 110. In some embodiments, system 10 is constructed and arranged to perform a surface modification to fiber matrix 110 and/or spine 210 that includes a modification of the surface energy of fiber matrix 110 and/or spine 210, respectively. In some embodiments, the surface of spine 210 is modified with a heated die comprising a textured or otherwise non-uniform surface. In some embodiments, fiber application assembly 400 and/or another component of system 10 comprise a radiofrequency plasma glow discharge assembly constructed and arranged to perform a surface modification of spine 210, such as a process performed in the presence of a material selected from the group consisting of: hydrogen; nitrogen; ammonia; oxygen; carbon dioxide; C2F6; C2F4; C3F6; C2H4; CH4; and combinations of one or more of these or other materials.

Supply 620 can comprise one or more of: a reservoir of one or more agents, such as agent 502; a power supply such as a laser power supply; and/or a reservoir of compressed fluid. In some embodiments, modifying element 627 comprises a nozzle, such as a nozzle configured to deliver a fiber matrix 110 modifying agent, a tubular conduit 120 modifying agent, a spine 210 modifying agent, and/or a graft device 100 modifying agent.

For clarification, any reference to a “nozzle” or “assembly”, in singular or plural form, can include one or more nozzles, such as one or more nozzles 427 or one or more modification elements 627 configured as a nozzle, or one or more assemblies, such as one or more polymer delivery assemblies 405 or one or more modification assemblies 605.

In some embodiments, modifying element 627 is configured to deliver an agent 502. For example, agent 502 can comprise a wax, a gel (e.g. a pluronic gel or other poloxamer gel) and/or other protective material delivered to tubular conduit 120 prior to the application of fiber matrix 110 to tubular conduit 120, the delivered agent 502 configured to protect tubular conduit 120 from adverse effects of solvent 52. Alternatively or additionally, agent 502 can comprise a neutralizing material (e.g. a material configured to neutralize adverse effects of solvent 52 as described herein), the agent 502 delivered to tubular conduit 120 prior to and/or during the application of fiber matrix 110 to tubular conduit 120. This delivery of agent 502 can be performed to prevent or otherwise minimize exposure of tubular conduit 120 to one or more solvents 52 (e.g. HFIP) included in polymer solution 50, and/or to reduce injury to tubular conduit 120 by solvent 52.

An agent 502 comprising a solvent-reducing material and/or a solvent neutralizing material can be delivered via mandrel 250 (e.g. when mandrel 250 comprises a porous mandrel), via modifying element 627, and/or via a separate device. Agent 502 can be applied to one or more surfaces of tubular conduit 120 via a method selected from the group consisting of: spraying; dipping; dripping; brushing, and combinations of one or more of these. Agent 502 can be applied to tubular conduit 120 prior to and/or after placing tubular conduit 120 around mandrel 250. In some embodiments, agent 502 comprises a solvent-reducing material comprising a thermogelling fluid, such as pluronic 407 poloxamer gel, or an equivalent, configured as a barrier. An agent 502 comprising a thermogel can be applied at a temperature below the solution gelation temperature such that the solution is in a liquid state during application, and subsequently gels on a surface of tubular conduit 120. Alternatively, the thermogel can be gelled prior to application onto tubular conduit 120. In some embodiments, an agent 502 comprising a gel or other material (e.g. a solvent-reducing material and/or a solvent neutralizing material) is applied at a thickness between 0.1 mm and 2 mm to one or more surfaces (e.g. the entire or a majority of the outer surface) of tubular conduit 120.

Agent 502 can comprise a thermogel solution prepared using distilled or ionized water, or a thermogel prepared using a preservative solution (e.g. to increase the buffering capacity of the thermogel). Examples of applicable preservative solutions include but are not limited to: phosphate buffered saline (PBS); cell culture media (e.g. Dulbecco's Modified Eagle Media or Gibco RPMI 1640); balanced salt solution (e.g. lactated ringer's solution or Hank's Balanced Salt solution); and/or a cardioplegia solution. Agent 502 can further comprise one or more materials added to a thermogel solution, such as to perform a function selected from the group consisting of: increase buffering capacity of the solution; modify the pH of the solution; act as a solvent scavenger (e.g. an HFIP scavenger); and combinations of one or more of these. For example, agent 502 can further comprise: a salt (e.g. a sodium or potassium salt); sodium bicarbonate; powdered cell culture media; uridine diphosphate glucuronic acid; and combinations of one or more of these. Following application of fiber matrix 110 onto tubular conduit 120, agent 502 can be left in place during implantation of device 100. Alternatively, device 100 can be placed in a solution (e.g. a cooled vein preservation solution), to re-liquefy agent 502 (e.g. re-liquefy a thermogel material component of agent 502) or otherwise treat agent 502 such that it can be removed from device 100.

Application of agent 502 (e.g. a poloxamer gel) onto a surface (e.g. the outer surface) of tubular conduit 120 as a temporary layer between fiber matrix 110 and tubular conduit 120 provides numerous advantages. In some embodiments, agent 502 comprising a gel or other material can provide an adhesive connection between tubular conduit 120 and fiber matrix 110, such as to improve post-application handling of tubular conduit 120. In some embodiments, agent 502 is applied as a temporary layer on the inner surface of tubular conduit 120, with sufficient thickness to allow a smaller diameter mandrel 250 to be used. In these embodiments, trauma to tubular conduit 120 (e.g. a vein) can be reduced.

In some embodiments, modifying element 627 is configured to deliver a kink resisting element, for example spine 210, such as when modifying element 620 comprises a robotic assembly constructed and arranged to laterally deliver spine 210 about at least conduit 120 (e.g. about conduit 120 and an inner layer of fiber matrix 110). Alternatively or additionally, modifying element 627 can be configured to modify conduit 120, spine 210 and/or fiber matrix 110, such as to cause graft device 100 to be kink resistant or otherwise enhance the performance of the graft device 100 produced by system 10. In these graft device 100 modifying embodiments, modifying element 627 can comprise a component selected from the group consisting of: a robotic device such as a robotic device configured to apply spine 210 to tubular conduit 120; a nozzle, such as a nozzle configured to deliver agent 502; an energy delivery element, such as a laser delivery element such as a laser excimer diode or CO2 laser, or another element configured to trim one or more components of graft device 100; a fluid jet, such as a water jet or air jet configured to deliver fluid during the application of fiber matrix 110 to conduit 120; a cutting element, such as a cutting element configured to trim spine 210 and/or fiber matrix 110; a mechanical abrader; and combinations of one or more of these or other components. Modification of fiber matrix 110 or other graft device 100 component by modifying element 627 can occur during the application of fiber matrix 110 and/or after fiber matrix 110 has been applied to conduit 120. Modification of one or more spines 210 can be performed prior to and/or after spine 210 has been applied to surround conduit 120. In some embodiments, modifying element 627 can be used to cut or otherwise trim fiber matrix 110 and/or a spine 210.

Modification assembly 605 of system 10 can be an additional component or assembly, separate from fiber application assembly 400, such as a handheld device configured to remove solvent 52 and/or deliver spine 210. In some embodiments, modification assembly 605 comprises a handheld laser, such as a laser device which can be hand operated by an operator. In some embodiments, modification assembly 605 comprises a separate (e.g. handheld) device including a fan, vacuum and/or other gas propelling device configured to remove solvent 52 from areas surrounding tubular conduit 120 and/or fiber matrix 110. Modification assembly 605 can be used to modify graft device 100 after removal of graft device 100 from fiber application assembly 400, such as just prior to and/or during an implantation procedure.

Laser-based, heat-based, cold-based, and/or other modifications to fiber matrix 110 can cause portions of fiber matrix 110 to undergo physical changes, such as hardening, softening, melting, stiffening, creating a resilient bias, expanding, and/or contracting. Alternatively or additionally, these modifications can cause fiber matrix 110 to undergo chemical changes, such as forming chemical bonds with an adhesive layer between the outer surface of conduit 120 and fiber matrix 110 and/or a chemical change that reduces the amount of solvent in fiber matrix 110. In some embodiments, modifying element 627 is alternatively or additionally configured to modify tubular conduit 120, such that tubular conduit 120 comprises a kink resisting or other performance enhancing element. Modifications to tubular conduit 120 can include but are not limited to a physical change (e.g. to one or more portions of tubular conduit 120) selected from the group consisting of: drying; hardening; softening; melting; stiffening; creating a resilient bias; expanding; contracting; and combinations of one or more of these or other changes. Modifications of tubular conduit 120 can cause tubular conduit 120 to undergo chemical changes, such as a chemical change that results in a reduction in solvent 52 in fiber matrix 110 and/or a chemical change that forms chemical bonds with an adhesive layer between an outer surface of conduit 120 and spine 210 and/or fiber matrix 110.

As described herein, fiber matrix 110 can include an inner layer and an outer layer, and the inner layer can include an adhesive component and/or exhibit adhesive properties. The inner layer can be delivered separate from the outer layer, for example, delivered from a separate nozzle or at a separate time during the process. Selective adhesion between the inner and outer layers can be included, and can be configured to provide kink resistance. Spine 210 can be placed between the inner and outer layers of fiber matrix 110, such as is described herein.

In some embodiments, fiber application assembly 400 can be configured to deliver fiber matrix 110, and/or an adhesive layer, according to set parameters configured to produce a kink resistant element in graft device 100 and/or provide kink resisting properties to graft device 100. For example, an adhesive layer can be delivered about conduit 120 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle 427 distance to mandrel 250 and/or conduit 120; linear travel distance of nozzle 427 and/or a fiber modifying element 627 along its respective drive assembly (for example, drive assembly 445 or 645); linear travel speed of nozzle 427 and/or a fiber modifying element 627 along its respective drive assembly; compositions of the polymer solution and/or adhesive layer; concentrations of the polymer solution and/or adhesive layer; solvent compositions and/or concentrations; fiber matrix 110 inner and outer layer compositions and/or concentrations; spontaneous or sequential delivery of the polymer solution 50 and the adhesive layer; voltage applied to the nozzle 427; voltage applied to the mandrel 250; viscosity of the polymer solution 50; temperature within chamber 20; relative humidity within chamber 20; airflow within chamber 20; and combinations of one or more of these or other parameters.

Nozzle 427 can be constructed of stainless steel, such as passivated 304 stainless steel. A volume of space surrounding nozzle 427 can be maintained free of objects or substances which can interfere with the electrospinning process. Nozzle 427 geometry and orientation, as well as the electrical potential voltages applied between nozzle 427 and mandrel 250 are chosen to control fiber generation.

Mandrel 250 is positioned in a particular spaced relationship from assembly 405 and/or assembly 605, and nozzle 427 and/or modifying element 627, respectively. In the illustrated embodiment, mandrel 250 is positioned above and below assemblies 605 and 405, respectively. Alternatively, mandrel 250 can be positioned either above, below, to the right and/or to the left of assembly 405 and/or assembly 605. The distance between mandrel 250 and the tip of nozzle 427 and/or modifying element 627 can be less than 20 cm, or less than 15 cm, such as a distance of between 12.2 cm and 12.8 cm, or a distance of approximately 12.5 cm. In some embodiments, multiple nozzles 427 and/or multiple modifying elements 627, for example components of similar or dissimilar configurations, can be positioned in various orientations relative to mandrel 250. In some embodiments, the distance between nozzles 427 and/or modifying elements 627 and mandrel 250 varies along the length of their respective travel along mandrel 250, such as to create a varying pattern of fiber matrix 110 along conduit 120. In some embodiments, nozzle 427 and/or modifying element 627 distances from mandrel 250 can vary continuously during the fiber application (e.g. electrospinning) process and/or the distance can vary for one or more set periods of time during the process.

In some embodiments, an electrical potential is applied between nozzle 427 and one or both of conduit 120 and mandrel 250 (e.g. when fiber application assembly 400 comprises an electrospinning device). The electrical potential can draw at least one fiber from polymer delivery assembly 405 to conduit 120. Conduit 120 can act as the substrate for an electrospinning or other fiber delivery process, collecting the fibers that are drawn from polymer delivery assembly 405 (e.g. via the applied electrical potential). In some embodiments, mandrel 250 and/or conduit 120 has a lower voltage than nozzle 427 to create the desired electrical potential. For example, the voltage of mandrel 250 and/or conduit 120 can be a negative or zero voltage while the voltage of nozzle 427 can be a positive voltage. Mandrel 250 and/or conduit 120 can have a voltage of about: −5 kV, −10 kV, −9 kV, −8 kV, −7 kV, −6 kV, −5 kV, −4.5 kV, −4 kV, −3.5 kV, −3.0 kV, −2.5 kV, −2 kV, −1.5 kV, or −1 kV. The nozzle 427 can have a voltage of about: +15 kV, 2.5 kV, 5 kV, 7.5 kV, 12 kV, 13.5 kV, 15 kV, 17 kV, or 20 kV. In some embodiments, the potential difference between nozzle 427 and mandrel 250 and/or conduit 120 can be from about 5 kV to about 30 kV. This potential difference draws fibers from nozzle 427 to conduit 120. In some embodiments, nozzle 427 is electrically charged with a potential of between about +15 kV and about +17 kV while mandrel 250 is at a potential of approximately −2 kV. In some embodiments, mandrel 250 is a fluid mandrel, such as the fluid mandrel described in applicant's co-pending U.S. patent application Ser. No. 13/997,933, filed Jun. 25, 2013, the content of which is incorporated herein by reference in its entirety for all purposes.

Mandrel 250 can be configured to rotate about an axis, such as central axis 435 of mandrel 250, with axis 428 of nozzle 427 typically oriented orthogonal to axis 435. In some embodiments, axis 428 of nozzle 427 is horizontally offset from axis 435. The rotation around axis 435 allows fiber matrix 110 to be applied along all sides, or around the entire circumference of conduit 120. In some embodiments, two motors 440a and 440b are used to rotate mandrel 250. Alternatively, fiber application assembly 400 can include a single motor configured to rotate mandrel 250, such as is described hereabove. The rate of rotation of mandrel 250 can determine how fibers (e.g. electrospun fibers) are applied to one or more segments of conduit 120. For example, for a thicker portion of fiber matrix 110, the rotation rate can be slower than when a thinner portion of fiber matrix 110 is desired. In some embodiments, mandrel 250 is rotated at a rate (e.g. a minimum, maximum or average rate) of between about 100 rpm and about 500 rpm, such as a rate of between about 200 rpm and about 300 rpm, between about 240 rpm and about 260 rpm, or approximately 250 rpm.

In addition to mandrel 250 rotating around axis 435, the polymer delivery assembly 405 can move, such as when driven by drive assembly 445 in a reciprocating or oscillating horizontal motion (to the left and right of the page). Drive assembly 445, as well as drive assembly 645 which operably attaches to assembly 605, can each comprise a linear drive assembly, such as a belt-driven and/or gear-driven drive assembly comprising two or more pulleys driven by one or more stepper motors. Additionally or alternatively, assemblies 405 and/or 605 can be constructed and arranged to rotate around axis 435, a rotation element not shown (e.g. with or without rotation of mandrel 250). The length of drive assemblies 445 and/or 645 and the linear motion applied to assemblies 405 and 605, respectively, can vary based on the length of conduit 120 to which a fiber matrix 110 is delivered and/or a fiber matrix 110 modification is applied. For example, the supported linear motion of drive assemblies 445 and/or 645 can comprises a translation distance of between about 10 cm to about 50 cm, such as to cause a translation of assembly 405 and/or 605 between about 25 cm and about 35 cm, between about 26 cm and about 32 cm, between about 27 cm and about 31 cm, or approximately 29 cm. Rotational speeds of mandrel 250, rotational speeds of assemblies 405 and/or 605, and/or translational speeds of assemblies 405 and/or 605 can be relatively constant, or they can be varied during the fiber application and/or modification process. In some embodiments, assembly 405 and/or 605 are translated (e.g. back and forth) at a relatively constant translation rate between about 40 mm/sec and about 150 mm/sec, such as to cause nozzle 427 and/or modifying element 627 to translate at a rate of between about 50 mm/sec and about 80 mm/sec, between about 55 mm/sec and about 65 mm/sec, or approximately 60 mm/sec, during the majority of its travel. In some embodiments, system 10 is constructed and arranged to rapidly change directions of translation (e.g. by maximizing deceleration before a direction change and/or maximizing acceleration after a direction change).

Assemblies 405 and/or 605 can move along the entire length and/or along specific portions of the length of conduit 120. In some embodiments, fiber is applied and/or a modification to the fiber matrix 110 is performed to the entire length of conduit 120 plus an additional length, such as 5 cm on either or both ends of conduit 120. In another embodiment, fiber(s) and/or a modification is applied to the entire length of conduit 120 plus at least 1 cm beyond either or both ends of conduit 120. Assemblies 405 and/or 605 can be controlled such that specific portions along the length of conduit 120 are reinforced with a greater amount (e.g. thicker segment) of fiber matrix 110 as compared to other or remaining portions. Alternatively or additionally, assemblies 405 and/or 605 can be controlled such that specific portions of the length of conduit 120 include one or more kink resistant elements (e.g. one or more spines 210) positioned at those one or more specific conduit 120 portions. In addition, conduit 120 can be rotating around axis 435 while assemblies 405 and/or 605 move, via drive assemblies 445 and/or 645, respectively, to position assemblies 405 and/or 605 at the particular portion of conduit 120 to which fiber is applied and/or modified.

System 10 can also include a power supply, power supply 410 configured to provide the electric potentials to nozzle 427 and mandrel 250, as well as to supply power to other components of system 10 such as drive assemblies 445 and 645 and assembly 605. Power supply 410 can be connected, either directly or indirectly, to at least one of mandrel 250 or conduit 120. Power can be transferred from power supply 410 to each component by, for example, one or more wires.

System 10 can include an environmental control assembly including environmental chamber 20 that surrounds fiber application assembly 400. System 10 can be constructed and arranged to control the environmental conditions within chamber 20, such as to control one or more areas surrounding polymer delivery assembly 405 and/or mandrel 250 during the application of fiber matrix 110 to conduit 120. Chamber 20 can include inlet port assembly 21 and outlet port assembly 22. Inlet port assembly 21 and/or outlet port assembly 22 can each include one or more components such as one or more components selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations of these. Chamber 20 can include one or more environmental control components that can monitor and/or control temperature, humidity and/or pressure within chamber 20 (e.g. one or more environmental control components controlled by environmental controller 35). Chamber 20 can be constructed and arranged to provide relatively uniform ventilation about mandrel 250 (e.g. about tubular conduit 120, fiber matrix 110 and/or spine 210) including an ultra-dry (e.g. 2 ppm water or other liquid content) compressed gas (e.g. air) source configured to reduce humidity within chamber 20. Inlet port assembly 21 and outlet port assembly 22 can be oriented to purge air from the top of chamber 20 to the bottom of chamber 20 (e.g. to remove vapors of one or more solvents such as HFIP, which can tend to settle at the bottom of chamber 20). Chamber 20 can be constructed and arranged to replace the internal volume of chamber 20 at least once every 3 minutes, once every 1 minute, or once every 30 seconds. Outlet port assembly 22 can include one or more filters 24 (e.g. replaceable cartridge filters) which are suitable for retaining solvent 52 (e.g. by filtering vapor including solvent 52) or other undesired materials evacuated from chamber 20. Alternatively or additionally, inlet port assembly 21 can include one or more filters 23 which are similarly suitable for retaining solvent 52 or other undesired materials delivered into chamber 20. Chamber 20 can be constructed and arranged to maintain a flow rate of gas through chamber 20 of at least about 30 liters per minute (L/min), at least about 40 L/min, at least about 45 L/min, at least about 50 L/min, at least about 55 L/min, or at least about 60 L/min, such as during an initial purge procedure. Subsequent to an initial purge procedure, a flow rate of at least about 5 L/min, at least about 10 L/min, at least about 15 L/min, at least about 20 L/min, at least about 25 L/min, or at least about 30 L/min can be maintained, such as to maintain a constant humidity level (e.g. a relative humidity between about 20% and about 24%). Chamber 20 can be further constructed and arranged to control temperature, such as to control temperature within chamber 20 to a temperature between about 15° C. and about 25° C., such as between about 16° C. and about 20° C. with a relative humidity between about 20% and about 24%, or between about 20 and about 22%, or between about 22% and about 24%, or between about 19% and about 25%. In some embodiments, one or more objects or surfaces within chamber 20 are constructed of an electrically insulating material and/or do not include sharp edges or exposed electrical components. In some embodiments, one or more metal objects positioned within chamber 20 are electrically grounded and/or maintained at a particular desired voltage level (e.g. a voltage level different than the voltage level of nozzle 427 and/or different than the voltage level of mandrel 250.

In some embodiments, system 10 is configured to produce a first graft device, graft device 100′ based on one or more component or process parameters. In these embodiments, graft device 100′ comprises tubular conduit 120′ and a fiber matrix 110′ applied by fiber application assembly 400 (e.g. an electrospinning device). Fiber matrix 110′ can be applied via polymer delivery assembly 405 supplied with polymer solution 50 at a flow rate of approximately 15 ml/hr. Fiber matrix 110′ can be applied when an electrostatic potential of approximately 17 kV is applied between nozzle 427 and mandrel 250, such as when nozzle 427 is charged to a potential of approximately +15 kV and mandrel 250 is charged to a potential of approximately −2 kV. Cumulative application time of fiber matrix 110′ can comprise an approximate time period of between about 11 minutes and about 40 seconds and about 17 minutes and about 30 seconds. The cumulative application time of fiber matrix 110′ can comprise a time period of approximately 11 minutes and 40 seconds when tubular conduit 120′ comprises an outer diameter of between approximately 3.4 mm and approximately 4.2 mm, a time period of approximately 14 minutes and 0 seconds when tubular conduit 120′ comprises an outer diameter between approximately 4.2 mm and approximately 5.1 mm, and/or a time period of approximately 17 minutes and 30 seconds when tubular conduit 120′ comprises an outer diameter between approximately 5.1 mm and approximately 6.0 mm.

Fiber matrix 110′ can comprise an average fiber size of approximately 7.8 μm, such as a population of fiber diameters with an average fiber size of approximately 7.8 μm with a standard deviation of 0.45 μm. Fiber matrix 110′ can comprise an average porosity of approximately 50.4%, such as a range of porosities with an average of 50.4% and a standard deviation of 1.1%. Fiber matrix 110′ can comprise a strength property selected from the group consisting of: stress measured at about 5% strain comprising a level between about 0.4 MPa and about 1.1 MPa; ultimate stress at a level of from about 4.5 MPa to about 7.0 MPa; ultimate strain at a level of from about 200% to about 400%; and combinations of these. Fiber matrix 110′ can comprise a compliance between approximately 0.2×10⁻⁴/mmHg and approximately 3.0×10⁻⁴/mmHg when measured in arterial pressure ranges. Fiber matrix 110′ can comprise an elastic modulus between about 10 megapascals (MPa) and about 15 MPa. Fiber matrix 110′ can be constructed and arranged with a targeted suture retention strength, such as an approximate suture retention strength of between about 2.0 Newtons (N) and about 4.0N with 6-0 Prolene™ suture (or equivalent) and/or between about 1.5N and about 3.0N with 7-0 Prolene™ suture (or equivalent). In some embodiments, graft device 100′ includes a spine 210′, such as a spine 210′ placed between inner and outer layers of fiber matrix 110′ (e.g. placed after one-third of the total thickness of fiber matrix 110′ is applied about conduit 120′).

In some embodiments, system 10 is configured to produce a second graft device, graft device 100″ based on one or more component or process parameters. In these embodiments, graft device 100″ comprises tubular conduit 120″ and a fiber matrix 110″ applied by fiber application assembly 400. Fiber matrix 110″ can be applied via polymer delivery assembly 405 supplied with polymer solution 50 at a flow rate of approximately 20 milliliter per hour (ml/hr). Fiber matrix 110″ can be applied when an electrostatic potential of approximately 19 kV is applied between nozzle 427 and mandrel 250, such as when nozzle 427 is charged to a potential of approximately +17 kV and mandrel 250 is charged to a potential of approximately −2 kV. Cumulative application time of fiber matrix 110″ can comprise an approximate time period of between 9 minutes and 30 seconds and 13 minutes and 40 seconds. The cumulative application time of fiber matrix 110″ can comprise a time period of approximately 9 minutes and 30 seconds when tubular conduit 120″ comprises an outer diameter between approximately 3.4 millimeters (mm) and approximately 4.2 mm; a time period of approximately 11 minutes and 30 seconds when tubular conduit 120″ comprises an outer diameter between approximately 4.2 mm and approximately 5.1 mm, and/or a time period of approximately 13 minutes and 40 seconds when tubular conduit 120″ comprises an outer diameter between approximately 5.2 mm and approximately 6.0 mm.

Fiber matrix 110″ can comprise an average fiber size of approximately 8.6 μm, such as a population of fiber diameters with an average fiber size of approximately 8.6 μm with a standard deviation of 0.45 μm. Fiber matrix 110″ can comprise an average porosity of approximately 46.9%, such as a range of porosities with an average of 46.9% and a standard deviation of 0.9%. Fiber matrix 110″ can comprise a strength property selected from the group consisting of: stress at about 5% strain comprising a level between about 0.6 MPa and about 1.3 MPa; ultimate stress at a level of from about 5.0 MPa to about 7.5 MPa; ultimate strain at a level of from about 200% to about 400%; and combinations of these. Fiber matrix 110″ can comprise a compliance between approximately 0.2×10⁻⁴/mmHg and approximately 3.0×10⁻⁴/mmHg when measured in arterial pressure ranges. Fiber matrix 110″ can comprise an elastic modulus between about 12 MPa and about 18 MPa. A fiber matrix may comprise an elastic modulus between about 10 MPa and about 20 MPa. A fiber matrix may comprise an elastic modulus between about 10 MPa and about 18 MPa. A fiber matrix may comprise an elastic modulus between about 12 MPa and about 20 MPa. A fiber matrix may comprise an elastic modulus between about 14 MPa and about 16 MPa. Fiber matrix 110″ can be constructed and arranged with a targeted suture retention strength, such as an approximate suture retention strength of between about 2.3N and about 4.3N with 6-0 Prolene™ suture and/or between about 2.0N and about 3.5N with 7-0 Prolene™ suture. A fiber matrix may comprise a suture retention strength of from about 1.0N to about 6.0N. A fiber matrix may comprise a suture retention strength of from about 2.0N to about 5.0N. A fiber matrix may comprise a suture retention strength of from about 1.0N to about 5.0N. A fiber matrix may comprise a suture retention strength of from about 2.0N to about 6.0N. A fiber matrix may comprise a suture retention strength of from about 2.0N to about 4.0N. A fiber matrix may comprise a suture retention strength of from about 2.0N to about 3.0N. In some embodiments, graft device 100″ includes a spine 210″, such as a spine 210″ placed between inner and outer layers of fiber matrix 110″ (e.g. placed after one-third of the total thickness of fiber matrix 110″ is applied about conduit 120″).

Fiber matrix 110′ and 110″ can comprise one or more similar features and/or one or more dissimilar features. Fiber matrix 110″ of graft device 100″ can comprise more bonds between fibers than fiber matrix 110′ of graft device 100′. The increased number of bonds can result in a higher fiber matrix 110″ density which can be configured to limit cellular infiltration into graft device 100″ (e.g. to increase the graft durability in vivo). Fiber matrix 110″ can comprise fibers that are flatter (i.e. more oval versus round) and/or denser than fibers of fiber matrix 110′. Fiber matrix 110″ can have a greater resiliency than fiber matrix 110′.

System 10 can comprise one or more solvent-reducing materials, such as solvent-reducing material 640 shown positioned within supply 620. In some embodiments, modification assembly 605 is configured to deliver solvent-reducing material 640 to tubular conduit 120 and/or fiber matrix 110 (e.g. onto tubular conduit 120 and/or fiber matrix 110). In some embodiments, solvent-reducing material comprises a material selected from the group consisting of: a desiccant; a material configured to bond with solvent 52; a material configured to absorb solvent 52; a neutralizing agent configured to neutralize solvent 52 (e.g. make less toxic or otherwise less harmful to the patient); and combinations of one or more of these. In some embodiments, solvent-reducing material 640 is delivered onto tubular conduit 120 to create a barrier (e.g. a barrier layer) between tubular conduit 120 and an applied layer of fiber matrix 110 comprising solvent 52. In some embodiments, solvent-reducing material 640 comprises a material selected from the group consisting of: desiccant; lipid; phospholipid; buffer; pH buffer; polyethylene; PTFE; fibrin; albumin; gelatin; oil; wax; PEG; carbon particle; activated carbon particle; alkaline material; powder; carbon particles; polymer beads; polymer gel; wicking fibrous membrane; solvent capillary transport system; ionizing gas; plasma; and combinations of one or more of these. In some embodiments, solvent-reducing material 640 comprises a pH buffer and/or alkaline material configured to prevent undesired pH changes in tubular conduit 120. In some embodiments, solvent-reducing material 640 comprises an ionizing gas configured to absorb or otherwise neutralize solvent 52. For example, a “cloud” of ionizing gas could be positioned proximate the tubular conduit 120 such that the polymer fibers delivered by polymer delivery assembly 405 pass through the ionizing gas and attenuate the negative effects of solvent 52.

In some embodiments, solvent-reducing material 640 comprises a material positioned as a barrier between tubular conduit 120 and fiber matrix 110. In some embodiments, solvent-reducing material 640 comprises a removable or otherwise temporary barrier (e.g. a barrier removed prior to implantation of graft device 100 in the patient). In some embodiments, solvent-reducing material 640 is applied to a surface of the tubular conduit 120 and/or the fiber matrix 110 (e.g. an inner layer of the fiber matrix 110). In some embodiments, solvent-reducing material 640 is delivered to tubular conduit 120 and/or fiber matrix 110 during application of polymer fibers by polymer delivery assembly 405. In some embodiments, solvent-reducing material 640 comprises a material configured to neutralize solvent 52, such as neutralizing agent 641 described herebelow.

System 10 can comprise one or more solvent neutralizing materials, such as solvent neutralizing material 641 shown positioned within supply 620. Solvent neutralizing material 641 can comprise a material configured to reduce injury to tubular conduit 120 by solvent 52 (e.g. when tubular conduit 120 comprises a vein segment or other living tissue). In some embodiments, modification assembly 605 is configured to deliver solvent neutralizing material 641 to tubular conduit 120 and/or fiber matrix 110 (e.g. onto tubular conduit 120 and/or fiber matrix 110), such as an application that occurs prior to the delivery of fiber matrix 110, during the delivery of fiber matrix 110 (e.g. delivered while polymer fibers are being applied, or delivered to a partial layer of fiber matrix 110 when no fibers are being applied) and/or after the delivery of fiber matrix 110. In some embodiments, solvent neutralizing material 641 comprises a material selected from the group consisting of: a buffer; polyethylene; polytetrafluoroethylene (PTFE); fibrin; albumin; gelatin; polyethylene glycol (PEG); carbon particle; activated carbon particle; sulfate; phosphate; adenosine diphosphate (ADP); adenosine triphosphate (ATP) converted from ADP; an acid reducing material; a lipid; a phospholipid; an acidophilic bacteria; an alkaliphilic bacteria; and combinations of one or more of these. In some embodiments, solvent neutralizing material 641 is positioned about at least a portion of tubular conduit 120 and/or an inner layer of fiber matrix 110 to function as a barrier to prevent interaction between solvent 52 and tubular conduit 120. In these barrier embodiments, the barrier can be configured to be removable (e.g. dissolvable or otherwise removable) prior to implantation of graft device 100 in the patient. In these barrier embodiments, solvent neutralizing material (and the resultant barrier) can comprise a material selected from the group consisting of: lipid; phospholipid; buffer; pH buffer; polyethylene; PTFE; fibrin; albumin; gelatin; oil; wax; PEG; carbon particle; activated carbon particle; alkaline material; powder; carbon particles; polymer beads; polymer gel; and combinations of one or more of these.

System 10 can comprise one or more solvent-reducing elements, such as solvent-reducing element 40 and/or solvent-reducing element 450. Solvent-reducing element 40, shown positioned in chamber 20, and solvent-reducing element 450, shown positioned in fiber application assembly 400, can comprise one or more devices or components configured to extract solvent 52, such as to extract solvent 52 from tubular conduit 120 (e.g. from the wall of tubular conduit 120), from fiber matrix 110 (e.g. from one or more layers of fiber matrix 110), and/or from locations surrounding these (e.g. one or more locations within chamber 20). Solvent-reducing element 40 and/or 450 can comprise a component selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations of one or more of these. Alternatively or additionally, solvent-reducing element 40 and/or 450 can comprise a solvent-reducing material, such as a material selected from the group consisting of: a desiccant; a material configured to bond with solvent 52; a material configured to absorb solvent 52; a material configured to neutralize solvent 52 (e.g. make less toxic or otherwise less harmful to the patient); and combinations of one or more of these.

In some embodiments, solvent-reducing element 40 and/or 450 comprise a fluid extraction element configured to reduce solvent 52, such as a vacuum applying element. In these embodiments, nozzle 427 and/or modifying element 627 can comprise the solvent-reducing element configured to extract fluid and/or apply a vacuum. In some embodiments, solvent-reducing element 40 and/or 450 comprise a temperature control element configured to reduce solvent 52, such as when environmental controller 35 adjusts or otherwise controls the temperature within chamber 20 to cause a reduction in solvent 52. In some embodiments, solvent-reducing element 40 and/or 450 comprise a fluid delivery element configured to deliver a gas or other fluid proximate tubular conduit 120 to remove solvent 52 (e.g. when nozzle 427 and/or modification element 627 comprise the solvent-reducing element delivering the fluid to enhance diffusion of solvent 52). In some embodiments, solvent-reducing element 40 and/or 450 comprise an agitating element, such as a fan or other agitating element proximate tubular conduit 120 (e.g. to create a stream of laminar or turbulent gas flow proximate tubular conduit 120). In some embodiments, solvent-reducing element 40 and/or 450 comprise a humidity control element configured to remove solvent 52. In some embodiments, solvent-reducing element 40 and/or 450 comprises at least a replaceable portion (e.g. a disposable portion used on a single patient only). In some embodiments, solvent-reducing element 40 and/or 450 comprise a translatable element, such as when nozzle 427 and/or modifying element 627 comprise the solvent-reducing element and are translated by linear drive assemblies 445 and/or 645, respectively. In some embodiments, solvent-reducing element 40 and/or 450 comprises one or more elements configured to rotate and/or translate relative to tubular conduit 120.

System 10 can comprise one or more sensors, such as one or more sensors configured to detect the presence or level of one or more solvents (e.g. sensors that produce a signal related to a solvent level), such as solvent 52. In some embodiments, chamber 20 comprises sensor 26 comprising one or more sensors. In some embodiments, controller 30 comprises sensor 36 comprising one or more sensors. In some embodiments, mandrel 250 comprises sensor 256 comprising one or more sensors (e.g. a sensor configured to measure a parameter of tubular conduit 120 such as a level of solvent 52). In some embodiments, polymer delivery assembly 405 comprises sensor 406 comprising one or more sensors. In some embodiments, fiber application assembly 400 comprises sensor 466 comprising one or more sensors. In some embodiments, modification assembly 605 comprises sensor 606 comprising one or more sensors. Sensor 26, 36, 256, 406, 466 and/or 606 can each comprise one or more sensors configured to measure a parameter (e.g. configured to produce a signal related to the level of a solvent 52) and produce a signal based on the measured parameter. In some embodiments, sensor 26, 36, 256, 406, 466 and/or 606 can be configured to measure the concentration or other amount of solvent 52 present within tubular conduit 120 (e.g. within a wall of tubular conduit 120), fiber matrix 110 and/or a location within chamber 20. System 10 can be configured to adjust one or more system parameters based on the sensor signal produced by sensor 26, 36, 256, 406, 466 and/or 606. In some embodiments, system 10 can be configured to alert an operator that the level of solvent 52 present in graft device 100 is below a threshold (e.g. to indicate that graft device 100 is ready for implantation based on a measured level of solvent 52 detected).

In some embodiments, sensor 26, 36, 256, 406, 466 and/or 606 comprise one or more sensors selected from the group consisting of: optical sensor; temperature sensor; humidity sensor; pH sensor; ganged litmus paper instrument; strain gauge; accelerometer; load cell; electrochemical sensor; pressure sensor; chemical sensor; a color changing chemical sensor; a photoionization sensor; fluorine sensor; a temperature sensor configured to measure cooling of the tubular conduit (e.g. to assess evacuation of solvent 52); a temperature sensor configured to measure the temperature between inlet port 21 and outlet port 22; a sensor configured to measure the weight of at least a portion of graft device 100; a sensor configured to measure the mass of at least a portion of graft device 100; a sensor configured to measure the acidity of at least a portion of graft device 100; a sensor configured to measure a parameter of the exhaust of chamber 20 (e.g. exhaust through outlet port 22); and combinations of one or more of these. System 10 can be configured to adjust one or more system parameters based on the one or more signals produced by one or more sensors 26, 36, 256, 406, 466 and/or 606. The one or more system parameters adjusted can comprise one or more parameters selected from the group consisting of: temperature proximate the tubular conduit; flow rate of fluid proximate the tubular conduit; rotation rate of the tubular conduit; translation rate of the tubular conduit; rotation rate of polymer delivery assembly; translation rate of the polymer delivery assembly; a nozzle to a mandrel distance; and combinations of one or more of these. The one or more system parameters can be adjusted prior to, during and/or after delivery of fiber matrix 110 to tubular conduit 120.

In some embodiments, sensor 26, 36, 256, 406, 466 and/or 606 comprise one or more sensors configured to produce a signal representing a solvent 52 parameter level (e.g. a solvent concentration or other quantitative assessment of the presence of solvent 52). System 10 can be configured to reduce solvent 52 until the solvent parameter level reaches a threshold (e.g. falls below a maximum level). For example, system 10 can be configured to perform a function selected from the group consisting of: maintaining graft device 100 within chamber 20; rotating the graft device 100 (e.g. rotating tubular conduit 120 and at least a portion of fiber matrix 110); providing a flow of gas proximate the graft device 100; providing an elevated temperature proximate the graft device 100; providing a desiccant proximate the graft device 100; and combinations of one or more of these.

In some embodiments, polymer delivery assembly 405 is configured to deliver fibers with an aspect ratio above 1 and/or to deliver hollow fibers, such that solvent 52 more rapidly evacuates the fiber. In some embodiments, polymer delivery assembly 405 is configured to deliver fibers with an aspect ratio between 1.01:1 and 10:1.

In some embodiments, system 10 comprises one or more functional elements, such as functional element 25 shown positioned on chamber 20. Functional element 25 can comprise an element configured to remove solvent 52 and/or to reduce the effects of solvent 52 on tubular conduit 120 (e.g. a vein segment or other living tissue). In some embodiments, functional element 25 comprises an element selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations of one or more of these. In alternative embodiments, functional element 25 comprises one or more elements positioned in fiber application assembly 400 or another component of system 10.

In some embodiments, system 10 is configured to reduce solvent 52 by rotating the tubular conduit 120. For example, system 10 can be configured to perform a rotation with or without the simultaneous delivery of fibers to tubular conduit 120, such as by rotating at an increased rotational velocity during delivery of fibers, and/or a rotation that occurs while no fibers are delivered by polymer delivery assembly 405 (e.g. a rotation after delivery of fiber matrix 110 is complete). In some embodiments, system 10 is configured to rotate tubular conduit at a minimum velocity (e.g. a constant or variable rate that includes a rate greater than 250 rpm) for a minimum time period (e.g. longer than 1 second), in order to sufficiently reduce solvent 52. In some embodiments, system 10 is configured to rotate tubular conduit 120 at a first rate while fiber matrix 110 is being delivered by polymer delivery assembly 405, and to rotate tubular conduit 120 (e.g. and fiber matrix 110) at a second rate (e.g. a higher rate) after the delivery of fiber matrix 110 has been completed.

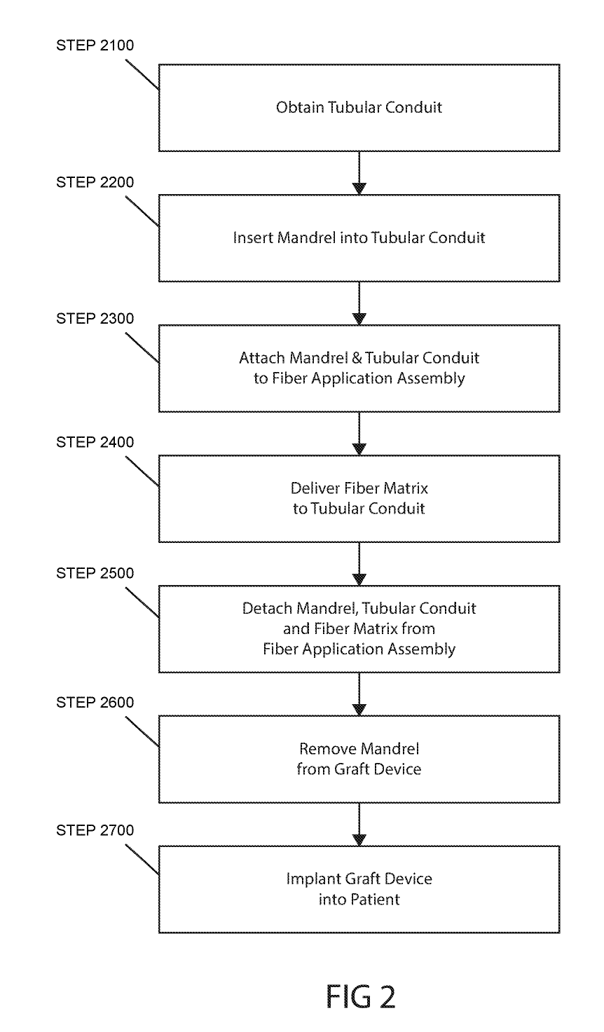

Referring now to FIG. 2, a flow chart of a method for producing and implanting a graft device is illustrated, consistent with the present inventive concepts. The method of FIG. 2 may be described using the components of system 10 described hereabove in reference to FIG. 1. In 2100, a tubular conduit 120 is obtained. In some embodiments, obtaining the tubular conduit 120 comprises harvesting tissue from a patient, such as harvesting a segment of vein or other tubular tissue.

In 2200, mandrel 250 is inserted into the tubular conduit 120.

In 2300, mandrel 250, including surrounding tubular conduit 120, is attached to rotating assembly 440 of fiber application assembly 400.

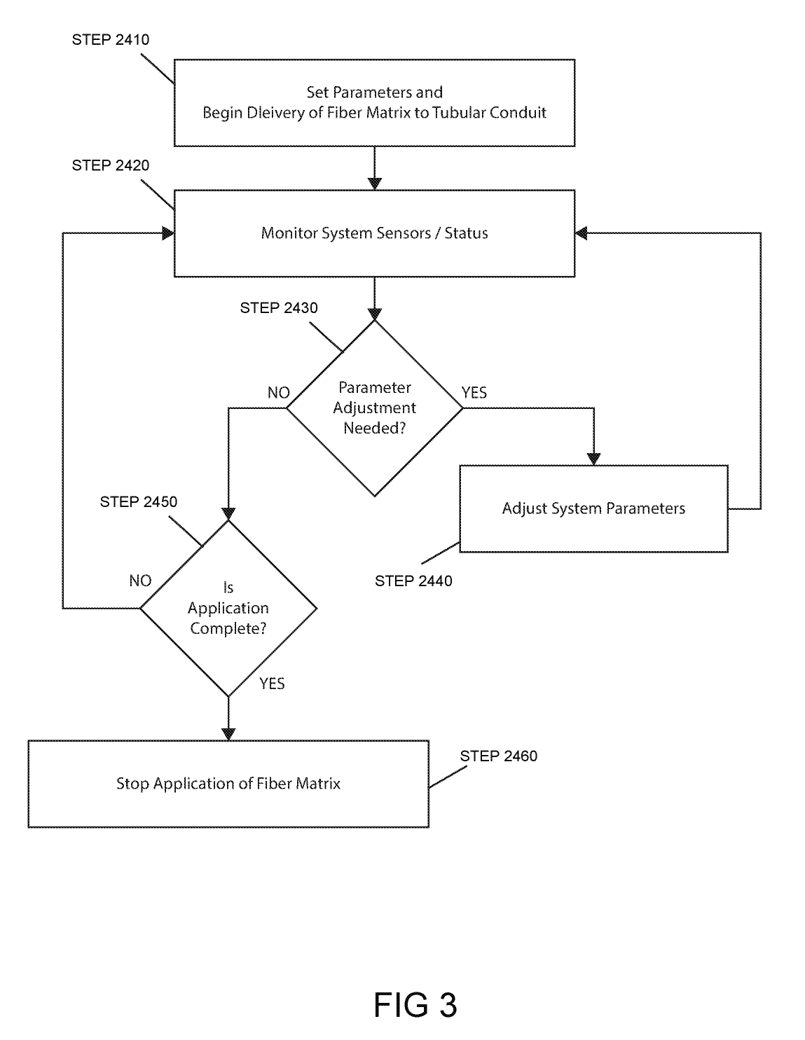

In 2400, polymer delivery assembly 405 delivers polymer fibers to tubular conduit 120 creating a graft device 100 comprising tubular conduit 120 surrounded, at least partially, by fiber matrix 110. During delivery of the polymer fibers to tubular conduit 120, mandrel 250 is rotated by rotating assembly 440 and polymer delivery assembly 405 is reciprocally translated by linear drive assembly 445. In some embodiments, one or more system parameters are adjusted while delivering the polymer fibers to tubular conduit 120, such as is described herebelow in reference to FIG. 3.

In 2500, mandrel 250 (still positioned within graft device 100) is detached from rotating assembly 440 of fiber application assembly 400.

In 2600, mandrel 250 is removed from graft device 100 (e.g. removed from tubular conduit 120).

In 2700, graft device 100 is implanted in the patient, such as by fluidly attaching first end 101 to a source of arterial blood (e.g. the aorta) and by fluidly attaching second end 102 to an artery (e.g. at a location downstream of an occlusion or other narrowing of the artery).

In some embodiments, a solvent-reducing process is performed, prior to, during and/or after one or more of Steps 2300, 2400, 2500, 2600 and/or 2700. For example, in some embodiments, a solvent-reducing process comprises providing gas flow (e.g. providing increased gas flow) proximate to fiber matrix 110 and/or tubular conduit 120 such as to cause removal of solvent 52 from fiber matrix 110 and/or tubular conduit 120. In some embodiments, a solvent-reducing process comprises a process selected from the group consisting of: providing gas flow or increasing gas flow proximate fiber matrix 110 and/or tubular conduit 120; elevating the temperature proximate fiber matrix 110 and/or tubular conduit 120; applying a reducing agent proximate fiber matrix 110 and/or tubular conduit 120; providing a vacuum proximate fiber matrix 110 and/or tubular conduit 120; and combinations of one or more of these.

In some embodiments, a solvent reduction process comprises a minimum waiting period, in which solvent 52 evacuates fiber matrix 110 and/or tubular conduit 120 such that an acceptable maximum level of solvent 52 remains (e.g. at or below an acceptable level for implantation into the patient). For example, system 10 can require that a pre-determined minimum time period has elapsed (e.g. after delivery of fiber matrix 110 is complete) before graft device 100 is implanted in the patient (as described hereabove in reference to 2700). For example, system 10 can activate an alert when the pre-determined time period has elapsed between the completion of 2500 and the initiation of 2700. Alternatively or additionally, mandrel 250 can remain in a “locked” condition for the minimum time period, and/or a door of chamber 20 can remain locked until the minimum time period has been exceeded. In these embodiments, the pre-determined time period can comprise a time period of at least about 2 minutes; about 5 minutes; about 7 minutes or about 10 minutes. In some embodiments, for at least a portion of the minimum time period, a solvent-reducing element (e.g. solvent-reducing element 40 and/or 450 described hereabove) is activated and/or positioned proximate tubular conduit 120 and/or fiber matrix 110 such as to remove solvent 52 from either or both.

In some embodiments, graft device 100, or at least tubular conduit 120, is positioned within a preservative and/or hydrating solution, such as a preservative solution comprising a material selected from the group consisting of: chilled fluid; fluid at approximately 4° C.; lactated ringers solution; papaverine; heparin; and combinations of one or more of these. For example, tubular conduit 120 can be positioned in a solution (e.g. a preservative solution) at any time between 2100 (e.g. a tissue harvesting procedure) and 2400 (delivery of the fiber matrix 110 onto the tubular conduit 120). Alternatively or additionally, graft device 100 can be positioned in a solution at any time between 2400 and 2700 (implantation of graft device 100 into the patient). In some embodiments, graft device 100 can be positioned in a solution during the minimum time period described hereabove. In some embodiments, one or more solutions such as a preservative and/or hydrating solution can be delivered by polymer delivery assembly 405 (e.g. via nozzle 427 or another fluid delivery element) or by modification assembly 605 (e.g. via modification element 627 or any fluid delivery element of modification assembly 605). In some embodiments, at least tubular conduit 120 can be positioned in a solution such as a hydrating or preservative solution described prior to and/or after the application of fiber matrix 110 to tubular conduit 120. In some embodiments, tubular conduit 120 (e.g. a vein or other harvested living tissue) is positioned in a preservative solution for at least 5 minutes, such as approximately 15 minutes, prior to application of fiber matrix 110 to tubular conduit 120 in 2400. Alternatively or additionally, graft device 100 can be positioned in a preservative solution for at least about 2 minutes, such as approximately 10 minutes, prior to implantation in the patient (e.g. after 2400 but before 2700).

In some embodiments, a solvent neutralizing process is performed prior to, during and/or after one or more of Steps 2300, 2400, 2500, 2600 and/or 2700. For example, in some embodiments, a solvent neutralizing process comprises delivering a solvent neutralizing agent (e.g. agent 641 described hereabove in reference to FIG. 1) onto fiber matrix 110 and/or tubular conduit 120 such as to reduce injury to tubular conduit 120 by solvent 52 (e.g. a neutralizing agent that chemically reacts with solvent 52 to make it inert and/or a neutralizing agent that acts as a barrier to prevent interaction of solvent 52 with tubular conduit 120).

Referring now to FIG. 3, a flow chart of a method for delivering polymer fibers to a tubular conduit while adjusting one or more system parameters is illustrated, consistent with the present inventive concepts. The method of FIG. 3 may be described using the components of system 10 described hereabove in reference to FIG. 1. In 2410, one or more system parameters are set, and polymer fibers are delivered to tubular conduit 120. In some embodiments, the system parameters comprise one or more parameters selected from the group consisting of: rotational velocity of mandrel 250; rotational velocity of polymer delivery assembly 405; translation rate of polymer delivery assembly 405 (e.g. by linear drive assembly 445); translation rate of modification assembly 605 (e.g. by linear drive assembly 645); translation rate of tubular conduit 120; flow rate of polymer solution 50 into polymer delivery assembly 405; voltage applied between nozzle 427 and mandrel 250; an environmental parameter of chamber 20 such as temperature within chamber 20, humidity within chamber 20, pressure within chamber 20, temperature proximate tubular conduit 120, humidity proximate tubular conduit 120 and/or pressure proximate tubular conduit 120; flow rate of air or other gas into chamber 20 via inlet port 21; flow rate of gas out of chamber 20 via outlet port 22; flow rate of air or other gas proximate tubular conduit 120; delivery of reducing agent 640 onto or otherwise proximate tubular conduit 120 and/or fiber matrix 110; nozzle 427 to tubular conduit 120 distance (e.g. distance to mandrel 250); modification element 627 to tubular conduit 120 distance (e.g. distance to mandrel 250); and combinations of one or more of these.

In 2420, one or more system parameters (e.g. as described hereabove) are monitored, such as by a sensor of system 10 such as by one, two or more of sensors 26, 36, 256, 406, 466 and/or 606 (e.g. when system 10 comprises at least one sensor or multiple sensors).

In 2430, system 10 performs an analysis (e.g. via an algorithm of controller 30) of a signal produced by one or more of sensors 26, 36, 256, 406, 466 and/or 606 and determines if a system parameter (e.g. as described hereabove) needs to be adjusted. For example, the signal produced by the sensor can correlate to an amount of solvent 52 present within tubular conduit 120, fiber matrix 110 and/or chamber 20 and the system parameter can be adjusted to reduce that amount of solvent.

If it is determined in 2430 that a system parameter needs to be adjusted, 2440 is performed in which the one or more identified system parameters (e.g. as described hereabove) are adjusted, and 2420 is subsequently performed.

If it is determined in 2430 that a system parameter doesn't need to be adjusted, 2450 is performed.

In 2450, an analysis is performed (e.g. via a timer or other algorithm of controller 30) to determine if the application of fiber matrix 110 to tubular conduit 120 is complete.

If it is determined that the application of fiber matrix 110 is not complete, 2420 is subsequently performed.

If it is determined that the application of fiber matrix 110 is complete, 2460 is performed in which the application of fiber matrix 110 is stopped (e.g. and subsequently mandrel 250 and graft device 100 are removed from fiber application assembly 400 as described hereabove in reference to FIG. 2). In some embodiments, system 10 is configured to prevent implantation of graft device 100 until a minimum time period has elapsed since application of fiber matrix 110 to conduit 120 is completed (e.g. via temporary locking of mandrel 250 and/or a door of chamber 20 as described herein).

While the graft devices herein have been described in detail as generally including an electrospun fiber matrix, other fiber delivery or other material application equipment can be used. Also, in some embodiments, the graft devices can include one or more spines or other kink resisting elements, or the applied fiber matrix can be configured to sufficiently resist kinking without the inclusion of the spine.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

We claim:

1. A system for applying a polymer fiber matrix to a tubular conduit to create a graft device, the system comprising:
a polymer solution comprising at least one polymer and at least one solvent;
a polymer delivery assembly constructed and arranged to receive the polymer solution and to deliver the polymer fiber matrix to the tubular conduit;
a rotating assembly constructed and arranged to rotate at least one of the tubular conduit or the polymer delivery assembly; and
a controller constructed and arranged to control the polymer delivery assembly and the rotating assembly;
wherein the system is constructed and arranged to reduce the amount of the solvent in the graft device.

2. The system according to claim 1, wherein the reducing of the amount of solvent is configured to reduce an event selected from the group consisting of: non-favorable healing response of tissue; delayed healing response of tissue; loss of tissue viability; and combinations thereof.

3. The system according to claim 1 or claim 2, wherein the system is configured to avoid implantation of the graft device during a minimum time period after delivery of the polymer fiber matrix to the tubular conduit.

4. The system according to claim 3, wherein the minimum time period comprises a duration of approximately 10 minutes.

5. The system according to claim 3, wherein the minimum time period comprises a duration selected from the group consisting of: about 2 minutes; about 5 minutes; about 7 minutes; and about 10 minutes.

6. The system according to claim 5, further comprising a preservative solution, wherein the tubular conduit comprises a blood vessel, and wherein the blood vessel is maintained in the preservative solution during the minimum time period.

7. The system according to claim 3, further comprising a solvent-reducing element constructed and arranged to be activated during the minimum time period and remove solvent from the graft device.

8. The system according to claim 7, wherein the solvent-reducing element comprises an element selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations thereof.

9. The system according to any one of claims 1-8, further comprising a chamber surrounding the tubular conduit during delivery of the polymer fiber matrix to the tubular conduit.

10. The system according to claim 9, wherein the chamber is constructed and arranged to be disposed of after the creation of the graft device.

11. The system according to claim 9, wherein the chamber is constructed and arranged to extract solvent from the delivered polymer fiber matrix during and/or after application of the polymer fiber matrix to the tubular conduit.

12. The system according to claim 9, wherein the chamber further comprises a filter constructed and arranged to remove solvent from the chamber.

13. The system according to claim 1, further comprising a solvent-reducing element constructed and arranged to reduce the amount of solvent in the graft device.

14. The system according to claim 13, wherein the solvent-reducing element comprises an element selected from the group consisting of: fan; nozzle; filter; electrostatic filter; osmotic membrane; fluid delivery element; fluid extraction element; vacuum applying element; agitating element; heating element; cooling element; sponge; diffusion enhancing element; desiccant; forced convection element; and combinations thereof.

15. The system according to claim 13, wherein the solvent-reducing element comprises a fluid extraction element constructed and arranged to remove fluid containing the solvent from the chamber during and/or after application of the polymer fiber matrix to the tubular conduit.

16. The system according to claim 13, wherein the solvent-reducing element comprises a temperature control element constructed and arranged to adjust temperature within the chamber to reduce the amount of solvent in the graft device.

17. The system according to claim 13, wherein the solvent-reducing element comprises a fluid delivery element constructed and arranged to deliver fluid into the chamber during and/or after application of the polymer fiber matrix to the tubular conduit.

18. The system according to claim 17, wherein the solvent-reducing element further comprises a fluid sprayed by the fluid delivery element to a location proximate the tubular conduit, wherein the fluid is configured to enhance diffusion.

19. The system according to claim 13, wherein the solvent-reducing element comprises an agitating element.

20. The system according to claim 19, wherein the agitating element comprises a fan.

21. The system according to claim 19, wherein the agitating element is positioned proximate the tubular conduit.

22. The system according to claim 19, wherein the agitating element comprises a stream of at least one of laminar gas flow or turbulent gas flow proximate the tubular conduit.

23. The system according to claim 13, wherein the solvent-reducing element comprises a humidity control element.

24. The system according to claim 13, wherein the solvent-reducing element comprises at least a replaceable portion.

25. The system according to claim 13, wherein the solvent-reducing element is constructed and arranged to translate and/or rotate about the tubular conduit.

26. The system according to claim 13, wherein the solvent-reducing element is configured to translate and/or rotate relative to the tubular conduit.

27. The system according to any one of claims 1-26, further comprising a solvent-reducing material.

28. The system according to claim 27, wherein the system is configured to apply the solvent-reducing material to the tubular conduit.

29. The system according to claim 27, wherein the solvent-reducing material comprises a solvent-absorbing material.

30. The system according to claim 27, wherein the solvent-reducing material comprises a material configured to chemically interact with the solvent.

31. The system according to claim 27, wherein the solvent-reducing material comprises a material selected from the group consisting of: desiccant; lipid; phospholipid; buffer; pH buffer; polyethylene; polytetrafluoroethylene (PTFE); fibrin; albumin; gelatin; oil; wax; polyethylene glycol (PEG); carbon particle; activated carbon particle; alkaline material; powder; carbon particles; polymer beads; polymer gel; wicking fibrous membrane; solvent capillary transport system; ionizing gas; plasma; and combinations thereof.

32. The system according to claim 27, wherein the solvent-reducing material is configured as a barrier for preventing interaction of solvent in the polymer fiber matrix with the tubular conduit.

33. The system according to claim 32, wherein the solvent-reducing material comprises a poloxamer gel.

34. The system according to claim 27, wherein the solvent-reducing material is constructed and arranged to be applied to at least one of the tubular conduit or the polymer fiber matrix.

35. The system according to claim 27, wherein the solvent-reducing material is constructed and arranged to be applied during application of the polymer fiber matrix to the tubular conduit.

36. The system according to claim 27, wherein the solvent-reducing material is constructed and arranged to be removed from at least one of the polymer fiber matrix or the tubular conduit prior to implantation of the graft device in a patient.

37. The system according to claim 27, wherein the system comprises a mandrel configured to be slidingly inserted into the tubular conduit, wherein the mandrel is further configured to deliver the solvent-reducing material to the tubular conduit.

38. The system according to claim 37, wherein the mandrel comprises a porous mandrel.

39. The system according to claim 27, wherein the system comprises a modifying element configured to deliver the solvent-reducing material to the tubular conduit.

40. The system according to claim 39, wherein the modifying element comprises a nozzle.

41. The system according to claim 27, wherein the polymer delivery assembly is configured to deliver the solvent-reducing material to the tubular conduit.

42. The system according to any one of claims 1-41, further comprising a sensor configured to produce a sensor signal, wherein the system is configured to reduce the amount of solvent based on the sensor signal.

43. The system according to claim 42, wherein the sensor comprises multiple sensors, each configured to produce a sensor signal.

44. The system according to claim 42, wherein the sensor comprises one or more sensors selected from the group consisting of: optical sensor; temperature sensor; humidity sensor; pH sensor; ganged litmus paper instrument; strain gauge; accelerometer; load cell; electrochemical sensor; pressure sensor; chemical sensor; color changing chemical sensor; a photoionization sensor; and combinations thereof.

45. The system according to claim 42, wherein the sensor comprises a fluorine sensor.

46. The system according to claim 42, wherein the sensor comprises a temperature sensor constructed and arranged to measure cooling of the tubular conduit.

47. The system according to claim 42, wherein the sensor comprises a sensor constructed and arranged to measure the temperature between an inlet port and an outlet port of the chamber.

48. The system according to claim 42, wherein the sensor comprises a sensor constructed and arranged to measure a parameter selected from the group consisting of: weight of the graft device; mass of the graft device; acidity of the graft device; a parameter of the exhaust of a chamber surrounding the tubular conduit during delivery of the polymer fiber matrix to the tubular conduit; and combinations thereof.

49. The system according to claim 42, wherein the system is configured to adjust a system parameter based on the sensor signal.

50. The system according to claim 49, wherein the adjusted system parameter comprises a parameter selected from the group consisting of: rotational velocity of a mandrel within the tubular conduit; rotational velocity of the polymer delivery assembly; translation rate of the polymer delivery assembly; translation rate of a modification assembly; translation rate of the tubular conduit; flow rate of the polymer solution into the polymer delivery assembly; voltage applied between a nozzle and a mandrel inserted into the tubular conduit; an environmental parameter of a chamber surrounding the tubular conduit such as temperature within the chamber, humidity within the chamber, pressure within the chamber, temperature proximate the tubular conduit, humidity proximate the tubular conduit and/or pressure proximate the tubular conduit; flow rate of air or other gas into a chamber surrounding the tubular conduit; flow rate of air or other gas proximate the tubular conduit; delivery of a reducing agent onto or otherwise proximate the tubular conduit and/or the polymer fiber matrix; distance between a nozzle and the tubular conduit; distance between a modification element and the tubular conduit; and combinations thereof.

51. The system according to claim 49, wherein the system is configured to adjust the system parameter at least one of: prior to; during; or after delivery of the polymer fiber matrix to the tubular conduit.

52. The system according to claim 42, wherein the sensor signal represents a solvent parameter level, and wherein the system is configured to reduce the amount of solvent in the graft device until the solvent parameter level reaches a threshold.

53. The system according to claim 52, wherein the reaching of the threshold comprises the solvent parameter level falling below a maximum level.

54. The system according to claim 52, wherein the system is configured to perform a function until the solvent parameter reaches a threshold, wherein the function is selected from the group consisting of: maintaining the graft device within a chamber of the system; rotating the graft device; providing a flow of gas proximate the graft device; providing an elevated temperature proximate the graft device; providing a desiccant proximate the graft device; and combinations thereof.

55. The system according to claim 42, wherein the system comprises a mandrel configured to be slidingly inserted into the tubular conduit, wherein the mandrel comprises the sensor.

56. The system according to any one of claims 1-55, wherein the polymer delivery assembly is constructed and arranged to deliver at least one of hollow fibers or flat fibers to the tubular conduit.

57. The system according to claim 56, wherein the polymer delivery assembly is constructed and arranged to deliver fibers comprising an aspect ratio between about 1.01:1 and about 10:1.

58. The system according to any one of claims 1-57, wherein the system is constructed and arranged to reduce the amount of solvent in the graft device by rotating the tubular conduit.

59. The system according to claim 58, wherein the system is configured to rotate the tubular conduit during delivery of the polymer fiber matrix to the tubular conduit.

60. The system according to claim 58, wherein the system is configured to rotate the tubular conduit after the delivery of the polymer fiber matrix to the tubular conduit is complete.

61. The system according to claim 85, wherein the system is configured to reduce the amount of solvent in the graft device by rotating the tubular conduit at a rate above a threshold for at least about 1 second.

62. The system according to claim 61, wherein the threshold comprises a rotational speed of at least about 250 rotations per minute (rpm).

63. The system according to claim 61, wherein the system is configured to rotate the tubular conduit at a variable rate.

64. The system according to claim 58, wherein the system is configured to rotate the tubular conduit for a first rate during the delivery of the polymer fiber matrix to the tubular conduit, and at a second rate after the delivery of the polymer fiber matrix to the tubular conduit.

65. The system according to claim 64, wherein the second rate is greater than the first rate.

66. The system according to any one of claims 1-65, wherein the graft device is constructed and arranged as a bypass graft.

67. The system according to any one of claims 1-66, wherein the graft device is constructed and arranged as a coronary artery bypass graft.

68. The system according to any one of claims 1-67, wherein the graft device is constructed and arranged as a peripheral artery bypass graft.

69. The system according to any one of claims 1-68, wherein the graft device is constructed and arranged as at least one of: a neo-artery or a neo-vein.

70. The system according to any one of claims 1-69, wherein the graft device further comprises a kink resisting element.

71. The system according to claim 70, wherein the kink resisting element is positioned between the tubular conduit and the polymer fiber matrix.

72. The system according to claim 70, wherein the polymer fiber matrix comprises an inner layer and an outer layer, and wherein the kink resisting element is positioned between the inner layer and the outer layer.

73. The system according to claim 70, wherein the kink resisting element is positioned outside of the polymer fiber matrix.

74. The system according to claim 70, wherein the kink resisting element comprises a spine.

75. The system according to claim 74, wherein the spine comprises a first support portion and a second support portion, wherein at least one of the first support portion or the second support portion is constructed and arranged to rotate relative to the other to receive the tubular conduit.

76. The system according to claim 74, wherein the spine comprises a first support portion comprising a first set of projections, and a second support portion comprising a second set of projections, wherein the first set of projections interdigitate with the second set of projections.

77. The system according to claim 70, wherein the kink resisting element comprises at least one filament comprising a diameter between about 0.4 millimeter (mm) and about 0.5 mm.

78. The system according to claim 70, wherein the kink resisting element comprises a resiliently biased element.

79. The system according to claim 70, wherein the kink resisting element is resiliently biased with a heat treatment.

80. The system according to claim 70, wherein the kink resisting element comprises a surface treated element.

81. The system according to claim 80, wherein the surface treated element increases a surface roughness of the kink resisting element.

82. The system according to any one of claims 1-81, wherein the polymer delivery assembly is configured to produce the polymer fiber matrix comprising a thickness between approximately 220 micrometer (μm) and approximately 280 μm.

83. The system according to any one of claims 1-82, wherein the polymer delivery assembly is configured to produce the polymer fiber matrix comprising fibers, wherein the fibers comprise a diameter between about 6 μm and about 15 μm.

84. The system according to claim 83, wherein the polymer delivery assembly is configured to produce the polymer fiber matrix comprising fibers, wherein the fibers comprise a diameter of approximately 7.8 μm.

85. The system according to claim 83, wherein the polymer delivery assembly is configured to produce the polymer fiber matrix comprising fibers, wherein the fibers comprise a diameter of approximately 8.6 μm.

86. The system according to any one of claims 1-85, wherein the polymer delivery assembly is configured to produce the polymer fiber matrix comprising a porosity between about 40% and about 80%.

87. The system according to claim 86, wherein the polymer delivery assembly is configured to produce the polymer fiber matrix comprising a porosity of approximately 46.9%.

88. The system according to claim 86, wherein the polymer delivery assembly is configured to produce the polymer fiber matrix comprising a porosity of approximately 50.4%.

89. The system according to any one of claims 1-88, wherein the polymer delivery assembly is configured to produce the polymer fiber matrix comprising a compliance between approximately 0.2×10⁻⁴/mmHg and approximately 3.0×10⁻⁴/mmHg.

90. The system according to any one of claims 1-89, wherein the polymer delivery assembly is configured to produce the polymer fiber matrix comprising an elastic modulus between about 10 megapascal (MPa) and about 18 MPa.

91. The system according to any one of claims 1-90, wherein the polymer delivery assembly comprises at least one nozzle.

92. The system according to claim 91, wherein the polymer delivery assembly is configured to deliver polymer solution to the at least one nozzle at a flow rate between about 10 milliliters per hour (ml/hr) and about 25 ml/hr.

93. The system according to claim 91, wherein the at least one nozzle comprises stainless steel.

94. The system according to claim 91, wherein the polymer delivery assembly further comprises a linear drive assembly configured to translate the at least one nozzle.

95. The system according to claim 94, wherein the linear drive assembly is configured to translate the at least one nozzle at least about 10 centimeters (cm).

96. The system according to any one of claims 1-95, wherein the tubular conduit comprises harvested tissue.

97. The system according to claim 96, wherein the harvested tissue comprises tissue selected from the group consisting of: saphenous vein; vein; artery; urethra; intestine; esophagus; ureter; trachea; bronchi; duct; fallopian tube; and combinations thereof.

98. The system according to any one of claims 1-97, wherein the tubular conduit comprises an artificial material.

99. The system according to claim 98, wherein the artificial material comprises a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations thereof.

100. The system according to any one of claims 1-99, wherein the polymer solution comprises a polymer weight to solvent volume ratio between about 20% and about 30%.

101. The system according to claim 100, wherein the polymer solution comprises a polymer weight to solvent volume ratio between about 24% and about 26%.

102. The system according to claim 101, wherein the polymer solution comprises a polymer weight to solvent volume ratio between about 24.5% and about 25.5%.

103. The system according to any one of claims 1-102, wherein the polymer solution comprises materials, wherein the materials comprise molecular weight averages between about 80,000 and about 150,000.

104. The system according to any one of claims 1-103, wherein the polymer solution comprises a viscosity between about 2000 centipoise (cP) and about 24000 cP.

105. The system according to any one of claims 1-104, wherein the polymer solution comprises a conductivity between about 0.4 microSiemens per centimeter (μS/cm) and about 1.70/cm.

106. The system according to any one of claims 1-105, wherein the polymer comprises a surface tension between about 21.5 milliNewton per meter (mN/m) and about 23.0 mN/m.

107. The system according to any one of claims 1-106, wherein the at least one polymer comprises at least two polymers.

108. The system according to claim 107, wherein the at least one polymer comprises a first polymer comprising a first hardness and a second polymer comprising a second hardness different than the first hardness.

109. The system according to claim 108, wherein the first material comprises a polyhexamethylene oxide soft segment.

110. The system according to claim 108, wherein the second material comprises an aromatic methylene diphenyl isocyanate hard segment.

111. The system according to any one of claims 1-110, wherein the at least one solvent comprises hexafluoroisopropanol.

112. The system according to claim 111, wherein the at least one solvent comprises HFIP, wherein the HFIP comprises about 99.97% minimum purity.

113. The system according to any one of claims 1-112, wherein the rotating assembly comprises at least one motor.

114. The system according to any one of claims 1-113, wherein the rotating assembly is configured to rotate at least one of the tubular conduit or the polymer delivery assembly at a rate between about 100 rpm and about 500 rpm.

115. The system according to 114, wherein the rotating assembly is configured to rotate at least one of the tubular conduit or the polymer delivery assembly at a rate between about 200 rpm and about 300 rpm.

116. The system according to 115, wherein the rotating assembly is configured to rotate at least one of the tubular conduit or the polymer delivery assembly at a rate between about 240 rpm and about 260 rpm.

117. The system according to any one of claims 1-116, wherein the rotating assembly is configured to rotate at least one of the tubular conduit or the polymer delivery assembly at a variable rate.

118. The system according to any one of claims 1-117, wherein the controller is configured to control a component selected from the group consisting of: the polymer delivery assembly; the rotating assembly; a linear drive assembly; a modification assembly; a voltage applied to a mandrel; and combinations thereof.

119. The system according to any one of claims 1-118, wherein the controller comprises an environmental controller.

120. The system according to claim 119, wherein the environmental controller is configured to remove solvent.

121. The system according to claim 119, wherein the environmental controller is configured to control one or more environmental parameters selected from the group consisting of: temperature; humidity; pressure; solvent concentration; and combinations thereof.

122. The system according to any one of claims 1-121, wherein the controller further comprises at least one gas propulsion mechanism.

123. The system according to claim 122, wherein the at least one gas propulsion mechanism comprises a fan.

124. The system according to any one of claims 1-123, wherein the controller is configured to detect an undesired state related to the solvent.

125. The system according to claim 124, wherein the controller comprises an alarm assembly.

126. The system according to claim 125, wherein the alarm assembly is configured to provide an alert selected from the group consisting of: audible alert; visual alert; tactile alert; and combinations thereof.

127. The system according to claim 124, wherein the controller is configured to stop delivery of the polymer fiber matrix to the tubular conduit when an undesired state is detected.

128. A system for applying a polymer fiber matrix to a tubular conduit to create a graft device, the system comprising:
a polymer delivery assembly constructed and arranged to receive a solution comprising polymer and solvent, and to deliver the polymer fiber matrix to the tubular conduit; and
a rotating assembly constructed and arranged to rotate at least one of the tubular conduit or the polymer delivery assembly;
a controller constructed and arranged to control the polymer delivery assembly and the rotating assembly;
wherein the system is constructed and arranged to reduce solvent-caused adverse effects to the tubular conduit of the graft device.

129. The system according to any one of claims 1-128, wherein the tubular conduit comprises living tissue.

130. The system according to claim 129, wherein the tubular conduit comprises living tissue selected from the group consisting of: saphenous vein; vein; artery; urethra; intestine; esophagus; ureter; trachea; bronchi; duct; fallopian tube; and combinations thereof.

131. The system according to any one of claims 1-130, further comprising a neutralizing agent configured to reduce the solvent-caused adverse effects.

132. The system according to claim 131, wherein the system is constructed and arranged to apply the neutralizing agent to the tubular conduit.

133. The system according to claim 132, wherein the system is constructed and arranged to apply the neutralizing agent at least one of: prior to; during; or after the delivery of the polymer fiber matrix to the tubular conduit.

134. The system according to claim 131, wherein the neutralizing agent comprises an agent selected from the group consisting of: a buffer; polyethylene; PTFE; fibrin; albumin; gelatin; PEG; carbon particle; activated carbon particle; sulfate; phosphate; adenosine diphosphate (ADP); adenosine triphosphate (ATP) converted from ADP; an acid reducing material; a lipid; a phospholipid; an acidophilic bacteria; an alkaliphilic bacteria; and combinations thereof.

135. The system according to claim 131, wherein the neutralizing agent is configured as a barrier surrounding the tubular conduit to prevent interaction between the solvent and the tubular conduit.

136. The system according to claim 135, wherein the barrier is configured to be removed prior to implantation of the graft device in the patient.

137. The system according to claim 135, wherein the barrier comprises a material selected from the group consisting of: lipid; phospholipid; buffer; pH buffer; polyethylene; PTFE; fibrin; albumin; gelatin; oil; wax; PEG; carbon particle; activated carbon particle; alkaline material; powder; carbon particles; polymer beads; polymer gel; a poloxamer gel; and combinations thereof.

138. The system according to claim 135, wherein the neutralizing agent comprises a poloxamer gel.

139. The system according to claim 131, wherein the system comprises a mandrel configured to be slidingly inserted into the tubular conduit, wherein mandrel is further configured to deliver the neutralizing agent to the tubular conduit.

140. The system according to claim 139, wherein the mandrel comprises a porous mandrel.

141. The system according to claim 131, wherein the system comprises a modifying element configured to deliver the neutralizing agent to the tubular conduit.

142. The system according to claim 141, wherein the modifying element comprises a nozzle.

143. The system according to claim 131, wherein the polymer delivery assembly is configured to deliver the neutralizing agent to the tubular conduit.

144. A method of creating a graft device comprising:
providing the system according to any claim herein; and
causing the polymer delivery assembly of the system to deliver the polymer fiber matrix to the tubular conduit.

145. The method according to claim 144, further comprising implanting the graft device in the patient after a minimum time period has elapsed since the delivery of the polymer fiber matrix to the tubular conduit.

146. The method according to claim 145, wherein the minimum time period comprises a duration selected from the group consisting of: about 2 minutes; about 5 minutes; about 7 minutes; and about 10 minutes.

147. The method according to claim 146, further comprising a preservative solution, wherein the tubular conduit comprises a blood vessel, and wherein the blood vessel is maintained in the preservative solution during the minimum time period.

148. The method according to any one of claims 144-147, wherein the tubular conduit comprises a vein segment, and wherein the method further comprises placing the vein in preservative solution.

149. The method according to claim 148, wherein the vein segment is placed in the preservative solution prior to delivering the polymer fiber matrix to the tubular conduit.

150. The method according to claim 149, further comprising placing the vein segment in the preservative solution after delivering the polymer fiber matrix to the tubular conduit.

151. The method according to claim 148, wherein the vein segment is placed in the preservative solution after delivering the polymer fiber matrix to the tubular conduit.

152. The method according to claim 148, wherein the preservative solution comprises a material selected from the group consisting of: chilled fluid; fluid at approximately 4° C.; lactated ringers solution; papaverine; heparin; and combinations thereof.
